(19) United States
(12) Patent Application Publication
(10) Pub. No.: US 2018/0044707 A1
(43) Pub. Date: Feb. 15, 2018

(54) Multi-Stage Enzymatic Hydrolysis of Lignocellulosic Biomass

(71) Applicants: Novozymes A/S, Bagsvaerd (DK); Beta Renewables Spa, Tortona (IT)

(72) Inventors: Jesper Frickmann, Raleigh, NC (US); Johan Belfrage, Cupar (GB); Eric Abbate, Vacaville, CA (US); David Osborn, Sacramento, CA (US); Geoffrey Moxley, Raleigh, NC (US); Daniele Riva, Prela Imperia (IT); Piero Otonello, Milano (IT); Simone Ferrero, Tortona (IT); Micol Purrotti, Condove (IT); Stefano Paravisi, Tortona (IT); Chiara Prefumo, Genova (IT)

(73) Assignees: Novozymes A/S, Bagsvaerd (DK); Beta Renewables Spa, Tortona (IT)

(21) Appl. No.: 15/557,300

(22) PCT Filed: Mar. 11, 2016
(86) PCT No.: PCT/US2016/022080

§ 371 Date: Sep. 11, 2017

Related U.S. Application Data
(60) Provisional application No. 62/132,126, filed on Mar. 12, 2015.

(51) Int. Cl. C12P 19/14; C12P 19/02

(52) U.S. Cl. CPC C12P 19/14; C12P 19/02; C12Y 302/01008; C12Y 302/01037; C12Y 302/01; C12P 2201/00

(57) ABSTRACT

The invention relates to processes of multi-stage hydrolysis where different enzyme compositions are added in at least two stages of hydrolysis. In a first stage, a first enzyme composition of xylanase, a beta-xylosidase and an endoglucanase is added, followed by a latter stage in which a second enzyme composition, a cellulolytic enzyme composition, is added. Also provided are processes for obtaining hydrolysis products and fermentation products using processes of the invention.

### REFERENCE TO A SEQUENCE LISTING

This application contains a Sequence Listing in computer readable form, which is incorporated herein by reference.

### FIELD OF THE INVENTION

The present invention relates generally to processes for enhancing enzymatic hydrolysis of biomass by conducting hydrolysis in at least two stages, where in a stage a first enzyme preparation comprising a combination of a xylanase, a beta-xylosidase and an endoglucanase is added, followed by a latter stage in which a second enzyme composition comprising cellulases is added. The invention also relates to processes for obtaining hydrolysis products and fermentation products using processes of the invention.

### DESCRIPTION OF RELATED ART

Renewable energy sources provide an alternative to current fossil fuel dependence. Production of ethanol as an energy source includes the basic steps of hydrolysis and fermentation. These steps are integrated within larger processes to obtain ethanol from various source materials.

Lignocellulosic biomass is comprised of cellulose, hemicellulose and lignin. To make the biomass accessible for hydrolysis, pretreatment is often performed, which may increase availability of the material for hydrolysis and thereby increase yields from hydrolysis processes. Selection of a pretreatment method may depend on many factors, such as biomass type, source and composition.

While pretreatment methods are effective to render the biomass available for hydrolysis, such methods may also generate inhibitors to hydrolysis and/or fermentation. Ideally, a selected pretreatment method will balance these considerations, maximizing availability of the biomass for hydrolysis, while minimizing formation of inhibitors.

In the hydrolysis step the source material is hydrolyzed to break down cellulose and/or hemicellulose to fermentable sugars. Commonly, enzymatic hydrolysis is utilized, but the presence of inhibitors, as well as other limitations may limit the yield achieved.

Hydrolysis processes may include batch reactors, continuously operating reactors, semi-batch reactors or semi-continuous reactors, or a combination thereof. Where the hydrolysis process includes use of, e.g., a mixed flow reactor, a fed batch reactor, or a continuously stirred tank reactor (CSTR), or series of such reactors, cost savings may be realized, as well as process advantages, such as ease of construction, increased volume production and decreased downtime.

However such systems may also provide limitations such as more detailed operations and possible problems arising from inefficient mixing within the reactor, as well as a startup time required to reach a steady state of operation.

Despite the potential limitations arising from selection of a reactor for hydrolysis, it is desired to boost production of fermentable sugars from the hydrolysis, while maintaining low overall expenditures of both time and resources.

While it is known that simply adding more enzyme during hydrolysis can often boost overall sugar production, and, correspondingly, fermentation yields, such an approach is not generally desirable in large scale production of ethanol, due to the increased costs of adding additional enzymes, as well as the possible inhibitory effects from accumulation of hydrolysis products, e.g., cellobiose, glucose and xylose.

There is therefore a need in the art for additional processes of hydrolyzing lignocellulosic biomass that address the inhibitors that may be present from pretreatment and improve the production of fermentable sugars and/or fermentation yields. Where continuously operating reactors are used in hydrolysis processes, there is a particular need for such improvement, without excessive increase in the total amount of enzymes or in enzyme consumption. The present invention provides such processes.

### SUMMARY OF THE INVENTION

Described herein are processes for hydrolyzing lignocellulosic material to improve yields of the resultant sugars for fermentation. The present invention is based on the surprising discovery that in a hydrolysis process comprising use of a continuous reactor, providing enzymes in a divided manner, as at least two different enzyme compositions, increases the yield of glucose and/or xylose in the resultant hydrolyzate as compared to adding all enzymes for enzymatic hydrolysis in a single stage hydrolysis. As such, the invention provides a multi stage hydrolysis process in which the enzyme compositions are added in separate stages.

In an aspect the hydrolysis includes use of a continuously operating reactor (e.g., continuously stirred tank reactor (CSTR)) and the sugar yield is increased to levels similar to those yields achieved in a pure batch process. Also described are processes for producing a fermentation product from the hydrolyzate of such a hydrolysis process.

Thus in one aspect, the invention relates to a process of improving a glucose or xylose yield of saccharification of a lignocellulosic material in a continuous reactor, the process comprising the steps of: a first stage comprising saccharifying a lignocellulosic material in a continuous reactor with a first enzyme composition comprising a xylanase, a beta-xylosidase and an endoglucanase; and a second stage comprising continuing saccharification of the lignocellulosic material, comprising combining the material of the first stage with a second enzyme composition comprising one or more cellulases to form a hydrolyzate, wherein the hydrolyzate has a glucose yield or a xylose yield that is improved as compared to the yield from a process comprising a single saccharification step. In another aspect the invention relates to a process of producing a fermentation product comprising fermentation of the hydrolyzate.

In another aspect the invention relates to a process of multi-stage hydrolysis of a lignocellulosic material, the process comprising the steps of: a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U, per gram of the lignocellulosic material and a second stage comprising continuing saccharification of the lignocellulosic material, comprising combining the material of the first stage with a second enzyme composition comprising one or more cellulases.

In a further aspect, the invention relates to a process of producing a fermentation product from a lignocellulosic material, the process comprising the steps of: hydrolyzing the lignocellulosic material, comprising: a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U per gram of the lignocellulosic material; and a second stage comprising continuing saccharification of the lignocellulosic material, comprising combining the material of the first stage with a second enzyme composition comprising one or more cellulases to form a hydrolyzate and fermenting the hydrolyzate to produce a fermentation product.

### Definitions

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more (e.g., several) alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST® 1.5L (Novozymes A/S, Bagsvaerd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can also be used in the presence of a soluble activating divalent metal cation according to WO 2008/151043 or WO 2012/122518, e.g., manganese or copper.

The AA9 polypeptide can be used in the presence of a dioxy compound, a bicylic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined as set forth in Example 10 herein.

Catalase: The term “catalase” means a hydrogen-peroxide:hydrogen-peroxide oxidoreductase (EC 1.11.1.6) that catalyzes the conversion of 2 H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme, cellulolytic composition, or cellulase: The term “cellulolytic enzyme,” “cellulolytic enzyme preparation”, “cellulolytic composition”, or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No1 filter paper, microcrystalline cellulose, bacterial cellulose, algal cellulose, cotton, pretreated lignocellulose, etc. The most common total cellulolytic activity assay is the filter paper assay using Whatman No1 filter paper as the substrate. The assay was established by the International Union of Pure and Applied Chemistry (IUPAC) (Ghose, 1987, Pure Appl. Chem. 59: 257-68). Cellulase activity can be determined as set forth in Example 8 herein.

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by an AMINEX® HPX-87H column (Bio-Rad Laboratories, Inc., Hercules, Calif., USA).

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, supra). Endoglucanase activity can also be determined using carboxymethyl cellulose (CMC) as substrate according to the procedure of Ghose, 1987, supra, at pH 5, 40° C.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 pmole of p-nitrophenolate anion per minute at pH 5, 25° C.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

High stringency conditions: The term “high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Hemicellulolytic enzyme, hemicellulolytic composition or hemicellulase: The term “hemicellulolytic enzyme”, “hemicellulolytic enzyme preparation,” “hemicellulolytic composition” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Homologous 3′ or 5′ region: The term “homologous 3′ region” means a fragment of DNA that is identical in sequence or has a sequence identity of at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a region in the genome and when combined with a homologous 5′ region can target integration of a piece of DNA to a specific site in the genome by homologous recombination. The term “homologous 5′ region” means a fragment of DNA that is identical in sequence to a region in the genome and when combined with a homologous 3′ region can target integration of a piece of DNA to a specific site in the genome by homologous recombination. The homologous 5′ and 3′ regions must be linked in the genome which means they are on the same chromosome and within at least 200 kb of one another.

Homologous flanking region: The term “homologous flanking region” means a fragment of DNA that is identical or has a sequence identity of at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a region in the genome and is located immediately upstream or downstream of a specific site in the genome into which extracellular DNA is targeted for integration.

Homologous repeat: The term “homologous repeat” means a fragment of DNA that is repeated at least twice in the recombinant DNA introduced into a host cell and which can facilitate the loss of the DNA, i.e., selectable marker that is inserted between two homologous repeats, by homologous recombination. A homologous repeat is also known as a direct repeat.

Host cell: The term “host cell” means any cell type that is susceptible to transformation, transfection, transduction, or the like with a nucleic acid construct or expression vector comprising a polynucleotide encoding a polypeptide. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. For instance, the mature polypeptide may be identified, using, e.g., the SignalP program (Nielsen et al., 1997, Protein Engineering 10: 1-6) that predicts a portion of the amino acid sequence as a signal peptide. As such, the mature polypeptide would be identified as the sequence lacking such redicted signal portion.

In one aspect, the mature polypeptide of a beta-glucosidase is amino acids 20 to 863 of SEQ ID NO: 2 based on the SignalP 3.0 program (Bendtsen et al., 2004, J. Mol. Biol. 340: 783-795) that predicts amino acids 1 to 19 of SEQ ID NO: 2 are a signal peptide. In another aspect, the mature polypeptide of a beta-glucosidase variant is amino acids 20 to 863 of SEQ ID NO: 4 based on the SignalP 3.0 program that predicts amino acids 1 to 19 of SEQ ID NO: 4 are a signal peptide. In another aspect, the mature polypeptide of a cellobiohydrolase I is amino acids 27 to 532 of SEQ ID NO: 6 based on the SignalP 3.0 program that predicts amino acids 1 to 26 of SEQ ID NO: 6 are a signal peptide. In another aspect, the mature polypeptide of a cellobiohydrolase II is amino acids 20 to 454 of SEQ ID NO: 8 based on the SignalP 3.0 program that predicts amino acids 1 to 19 of SEQ ID NO: 8 are a signal peptide. In another aspect, the mature polypeptide of In another aspect, the mature polypeptide of an AA9 polypeptide is amino acids 26 to 253 of SEQ ID NO: 10 based on the SignalP 3.0 program that predicts amino acids 1 to 25 of SEQ ID NO: 10 are a signal peptide. In another aspect, the mature polypeptide of a GH10 xylanase is amino acids 20 to 397 of SEQ ID NO: 12 based on the SignalP 3.0 program that predicts amino acids 1 to 19 of SEQ ID NO: 12 are a signal peptide. In another aspect, the mature polypeptide of a GH10 xylanase is amino acids 20 to 398 of SEQ ID NO: 14 based on the SignalP 3.0 program that predicts amino acids 1 to 19 of SEQ ID NO: 14 are a signal peptide. In another aspect, the mature polypeptide of a beta-xylosidase is amino acids 21 to 792 of SEQ ID NO: 16 based on the SignalP 3.0 program that predicts amino acids 1 to 20 of SEQ ID NO: 16 are a signal peptide. In another aspect, the mature polypeptide of a beta-xylosidase is amino acids 22 to 796 of SEQ ID NO: 18 based on the SignalP 3.0 program that predicts amino acids 1 to 21 of SEQ ID NO: 18 are a signal peptide. In another aspect, the mature polypeptide of an endoglucanase II is amino acids 19 to 335 of SEQ ID NO: 20 based on the SignalP 3.0 program that predicts amino acids 1 to 18 of SEQ ID NO: 20 are a signal peptide.

It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having enzyme activity.

Medium stringency conditions: The term “medium stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent Enzyme: The term “parent” means an enzyme to which an alteration is made to produce a variant. The parent may be a naturally occurring (wild-type) polypeptide or a variant thereof.

Pretreated cellulosic or hemicellulosic material: The term “pretreated cellulosic or hemicellulosic material” means a cellulosic or hemicellulosic material derived from biomass by treatment with heat and dilute sulfuric acid, alkaline pretreatment, neutral pretreatment, or any pretreatment known in the art.

Pretreated corn stover: The term “Pretreated Corn Stover” or “PCS” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence, wherein the subsequence encodes a fragment having enzyme activity. In one aspect, a subsequence contains at least 85%, e.g., at least 90% or at least 95% of the nucleotides of the mature polypeptide coding sequence of an enzyme.

Transformant: The term “transformant” means a cell which has taken up extracellular DNA (foreign, artificial or modified) and expresses the gene(s) contained therein.

Transformation: The term “transformation” means the introduction of extracellular DNA into a cell, i.e., the genetic alteration of a cell resulting from the direct uptake, incorporation and expression of exogenous genetic material (exogenous DNA) from its surroundings and taken up through the cell membrane(s).

Variant: The term “variant” means a polypeptide having enzyme or enzyme enhancing activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Very high stringency conditions: The term “very high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Whole broth preparation: The term “whole broth preparation” means a composition produced by a naturally-occurring source, i.e., a naturally-occurring microorganism that is unmodified with respect to the cellulolytic and/or hemicellulolytic enzymes produced by the naturally-occurring microorganism, or a non-naturally-occurring source, i.e., a non-naturally-occurring microorganism, e.g., mutant, that is unmodified with respect to the cellulolytic and/or hemicellulolytic enzymes produced by the non-naturally-occurring microorganism.

Wild-Type Enzyme: The term “wild-type” enzyme means an enzyme expressed by a naturally occurring microorganism, such as a bacterium, yeast, or filamentous fungus found in nature.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrmann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al., 1992, Interlaboratory testing of methods for assay of xylanase activity, Journal of Biotechnology 23(3): 257-270.

Xylan degrading activity can be determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined as set forth in Example 9 herein.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### BRIEF DESCRIPTION OF THE DRAWINGS

FIG. 1 is a graph showing the glucose yield of two hydrolysis samples as described in Example 1.

FIG. 2 is a graph of the measured viscosity of biomass slurry versus time for the three different enzyme dosages as described in Example 2.

FIG. 3 is a graph showing the MIN pressure measurements obtained with a ViPr viscometer for 5 mg EP/g glucan of each enzyme composition, as set forth in Example 3.

FIG. 4 is a graph showing the MIN pressure measurements obtained with a ViPr viscometer for varied doses (3, 4, 5, 6, 7 and 8 mg EP/g glucan) of enzyme composition CPrepA, as set forth in Example 3.

FIG. 5 is a graph showing the MIN pressure measurements obtained with a ViPr viscometer for varied doses (1.5, 2, 3, 4, 5, 6 mg EP/g glucan) of the 50:50 blend of XPrepB and EG1, as set forth in Example 3.

FIG. 6 provides graphs of the glucose yield (A)and xylose yield (B) from each of the four reactors described Example 5: R1: CPrepA, 2% DO, pH5.2; R2: EG1+XPrepB for 18.5 h, CPrepA 2% DO, pH5.2; R3: EG1+XPrepB for 18.5 h at pH 4.8, CPrepA 2% DO, pH5.2; R4: EG1+XPrepB for 18.5 h at pH 5.2, CPrepA 2% DO, pH5.2.

FIG. 7 provides graphs of the pH activity (A) and temperature activity (B) of the endoglucanase composition described in Example 6.

FIG. 8 provides a graph of the pH activity of the beta-xylosidase composition as described in Example 7.

### DETAILED DESCRIPTION

Described herein are processes for improving the yield of one or more sugars from a hydrolysis process, the process comprising administration of enzymes for hydrolysis as at least two different enzyme compositions. Further described are processes of hydrolysis and processes of fermentation incorporating such improved sugar yield processes. Also described are enzyme compositions suitable for use in the processes and/or methods described herein.

The present inventors have surprisingly found that by conducting hydrolysis in at least two stages, a first stage comprising contacting a pretreated lignocellulose-containing material with a first enzyme composition comprising a combination of xylanase, beta-xylosidase and an endoglucanase, followed by a latter stage in which a second enzyme composition comprising cellulases is added, the hydrolysis yield can be increased. In another embodiment, the first enzyme composition comprises at least an endoglucanase, followed by a latter stage in which a second enzyme composition comprising cellulases is added. In a further embodiment the multi stage hydrolysis comprises use of a continuous reactor in at least one stage.

Increase of the yield is achieved as compared to the yield obtained from an equivalent process not utilizing a multi-stage process as described herein. In one embodiment the hydrolysis yield is increased relative to a process in which all enzymes for enzymatic hydrolysis are added in a single stage. In another embodiment the hydrolysis yield is increased relative to a process in which all enzymes for enzymatic hydrolysis are blended prior to administration. In still another embodiment the hydrolysis yield is increased relative to a process in which all enzymes for enzymatic hydrolysis are added in a constant feed. In still another embodiment, the hydrolysis yield from a multi-stage hydrolysis process comprising use of a continuous reactor is increased to a level comparable to the yield obtained from a pure batch hydrolysis.

The first and second enzyme compositions are different from one another. In a particular embodiment a first enzyme composition comprises a combination of a xylanase, a beta-xylosidase and one or more cellulases. In a further embodiment the first enzyme composition comprises a combination of a xylanase, a beta-xylosidase and an endoglucanase. In a still further embodiment a first enzyme composition comprises at least an endoglucanase. Taken together, the first and second enzyme compositions provide up to about 100% of the total enzymes added during enzymatic hydrolysis. In an embodiment the first enzyme composition provides about 1 to about 99%, e.g., about 1% to about 45%, about 2% to about 40%, or about 5% to about 35% of the total enzyme protein added in hydrolysis.

### Cellulosic Material

Processes of the present invention are carried out using cellulosic material. The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material may be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred embodiment, the cellulosic material is any biomass material. In another preferred embodiment the cellulosic material is lignocellulose-containing biomass material. In another preferred embodiment, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, switchgrass, or wheat straw.

In one embodiment, the cellulosic material is fiber, such as corn fiber or wheat fiber. Fiber, such as corn or wheat fiber, may be obtained by fractionation. Fractionation technologies are well-known in the art. In one embodiment the cellulosic material is fiber obtained from dry fractionation processes. In one embodiment the cellulosic material is fiber obtained from wet fractionation processes.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass may be algae, emergent plants, floating-leaf plants, or submerged plants.

The cellulosic material may be used as is or may be subjected to pretreatment, using conventional methods known in the art, as described more fully herein. In a preferred embodiment, the cellulosic material is pretreated.

### Hemicellulosic Material

The term “hemicellulosic material” means any material comprising hemicelluloses. Hemicelluloses include xylan, glucuronoxylan, arabinoxylan, glucomannan, and xyloglucan. These polysaccharides contain many different sugar monomers. Sugar monomers in hemicellulose can include xylose, mannose, galactose, rhamnose, and arabinose. Hemicelluloses contain most of the D-pentose sugars. Xylose is in most cases the sugar monomer present in the largest amount, although in softwoods mannose can be the most abundant sugar. Xylan contains a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67. Hemicellulosic material is also known herein as “xylan-containing material”.

Sources for hemicellulosic material are essentially the same as those for cellulosic material described herein. It is understood herein that the hemicellulose may be in the form of lignocellulose, a plant cell wall material containing lignin, cellulose, and hemicellulose in a mixed matrix. In a preferred embodiment, the hemicellulosic material is any biomass material. In another preferred embodiment, the hemicellulosic material is lignocellulose, which comprises cellulose, hemicelluloses, and lignin.

### Pretreatment of Cellulosic Material

In practicing the processes of the present invention, the cellulosic material used may be pretreated by any pretreatment process known in the art, used to disrupt plant cell wall components of cellulosic or hemicellulosic material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic or hemicellulosic material may also be subjected to particle size reduction, sieving, pre-soaking, wetting, washing, and/or conditioning prior to or with additional pretreatment methods, using methods known in the art or as otherwise described herein.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In an embodiment the cellulosic or hemicellulosic material is pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, the cellulosic or hemicellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment: The term “chemical pretreatment” refers to any chemical pretreatment that promotes the separation and/or release of cellulose, hemicellulose, and/or lignin. Such a pretreatment may convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment may be performed with a number of reactor designs, e.g., plug-flow reactors, counter-current reactors, or continuous counter-current shrinking bed reactors (Duff and Murray, 1996, supra; Schell et al., 2004, Bioresource Technology 91: 179-188; Lee et al., 1999, Adv. Biochem. Eng. Biotechnol. 65: 93-115).

Several methods of pretreatment under alkaline conditions may also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, supra). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, supra, and U.S. Published Application 2002/0164730.

In one embodiment, the chemical pretreatment is preferably carried out as a dilute acid treatment, and more preferably as a continuous dilute acid treatment. The acid is typically sulfuric acid, but other acids may also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one embodiment, the acid concentration is in the range from preferably 0.01 to 10 wt. % acid, e.g., 0.05 to 5 wt. % acid or 0.1 to 2 wt. % acid. The acid is contacted with the cellulosic material and held at a temperature in the range of preferably 140-200° C., e.g., 165-190° C., for periods ranging from 1 to 60 minutes.

In another embodiment, pretreatment takes place in an aqueous slurry. In preferred embodiments, the cellulosic material is present during pretreatment in amounts preferably between 10-80 wt. %, e.g., 20-70 wt. % or 30-60 wt. %, such as around 40 wt. %. The pretreated cellulosic material may be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment may involve various types of grinding or milling (e.g., dry milling, wet milling, or vibratory ball milling).

The cellulosic material may be pretreated both physically (mechanically) and chemically. Mechanical or physical pretreatment may be coupled with steaming/steam explosion, hydrothermolysis, dilute or mild acid treatment, high temperature, high pressure treatment, irradiation (e.g., microwave irradiation), or combinations thereof. In one embodiment, high pressure means pressure in the range of preferably about 100 to about 400 psi, e.g., about 150 to about 250 psi. In another embodiment, high temperature means temperature in the range of about 100 to about 300° C., e.g., about 140 to about 200° C. In a preferred embodiment, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments may be carried out sequentially or simultaneously, as desired.

Accordingly, in a preferred embodiment, the cellulosic material is subjected to physical (mechanical) or chemical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques may involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Hydrolysis (Saccharification)

In the hydrolysis step (i.e., saccharification step) the cellulosic material, e.g., pretreated lignocellulose, is hydrolyzed to break down cellulose and/or hemicellulose to fermentable sugars, such as glucose, cellobiose, xylose, xylulose, arabinose, mannose, galactose, and/or soluble oligosaccharides. The hydrolysis is performed enzymatically by providing one or more enzyme compositions in one or more saccharification stages.

In conversion of biomass substrates to ethanol and other fuels, particularly in large scale operations, many factors may limit the resultant yield. Specifically addressing such limitations may allow an increase in yield from hydrolysis and, subsequently, fermentation.

While it is desirable in saccharification to provide efficient conversion of biomass to fermentable sugars, simply increasing the solids loading does not produce a corresponding increase in converted product. In fact, as the solids loading is increased, a decrease in enzymatic digestion is generally observed. Such decrease may be attributable to factors such as increased viscosity, difficulty of maintaining enzyme distribution, and increased generation of inhibitors.

In large scale biomass processing, handling of high solids is necessary. However, attempting to process high solids in a batchwise manner will result in a high viscosity, which may result in a slurry that is difficult to pump or stir or otherwise requiring additional means for handling. One method of addressing viscosity has been to operate in a continuous or semi-continuous manner in which the substrate and/or enzymes are fed to a reactor, continuously or periodically. Another manner of addressing viscosity has been shown by administering an endoglucanase for liquefaction prior to saccharification of a lignocellulosic material (WO 2014/108454).

However, even when the viscosity is reduced or otherwise addressed, inhibitors may provide a further hurdle to achieving high yields from hydrolysis. In use of hemicellulose-containing substrates, pretreatment and hydrolysis will result in generation of xylan and various xylooligomers. It is known that the presence of such can inhibit enzymatic activity and inhibit hydrolysis. It has been shown that supplementation with xylanase and beta-xylosidase in large amounts prior to addition of cellulases is beneficial in reducing these known inhibitors in a batch process. (Qing and Wyman, Biotechnology for Biofuels 2011, 4:18.)

The inventors have surprisingly discovered that by using a multi-stage hydrolysis with administration of enzymes as at least two separate enzyme compositions, hydrolysis yields in a hydrolysis process comprising a continuous reactor can be increased, without requiring large amounts of total enzyme protein. Such yields can be increased to levels similar to those achieved from a pure batch process.

Processing of cellulosic material according to the present invention can be implemented using any conventional biomass processing apparatus configured to operate in accordance with embodiments of the invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

The hydrolysis can be carried out as a continuous process, or series of batch and/or series of continuous processes, where the cellulosic or hemicellulosic material is fed gradually to, for example, an enzyme-containing hydrolysis solution. In an embodiment of the invention comprising a multi-stage hydrolysis, at least two stages are carried out in a single reactor. The hydrolysis may also be carried out as a series of batch and continuous processes.

Operation of multiple reactors in series allows for closer control of elements within each reactor, e.g., temperature, pH, mixing, concentration, and the like. Therefore in an embodiment of the invention comprising a multi-stage hydrolysis, at least two stages are carried out in separate reactors. In a preferred embodiment, each stage of a multi-stage hydrolysis is carried out in a separate reactor. In a further preferred embodiment, at least one stage in a multi-stage hydrolysis is carried out in a continuous reactor (e.g., CSTR). In a still further preferred embodiment, a continuous reactor in a multi-stage hydrolysis process is followed in series with at least one additional reactor. In another preferred embodiment, a CSTR in a multi-stage hydrolysis process is followed, in series with at least one additional reactor. In yet another preferred embodiment, a continuously stirred reactor is followed in series by at least one batch reactor.

Continuous operation, such as in use of a CSTR, provides advantages of continuous production and a steady state of operation once the reactor is running. Use of a continuous reactor permits management of high viscosity unhydrolyzed substrate, which also permits operation with higher total solids than might be available in a batch reactor. Semi-batch and semi-continuous operation may permit control of environmental conditions and provide additional flexibility, compared to pure batch processes for selection of optimal conditions. For large scale hydrolysis processes, continuous operation is often preferred to eliminate downtime and to maximize production, though semi-batch and semi-continuous operation may also be used. However, as provided in Example 1, a gap in performance may be seen via a reduced sugar yield when a CSTR is used in a hydrolysis process, versus using a pure batch reactor.

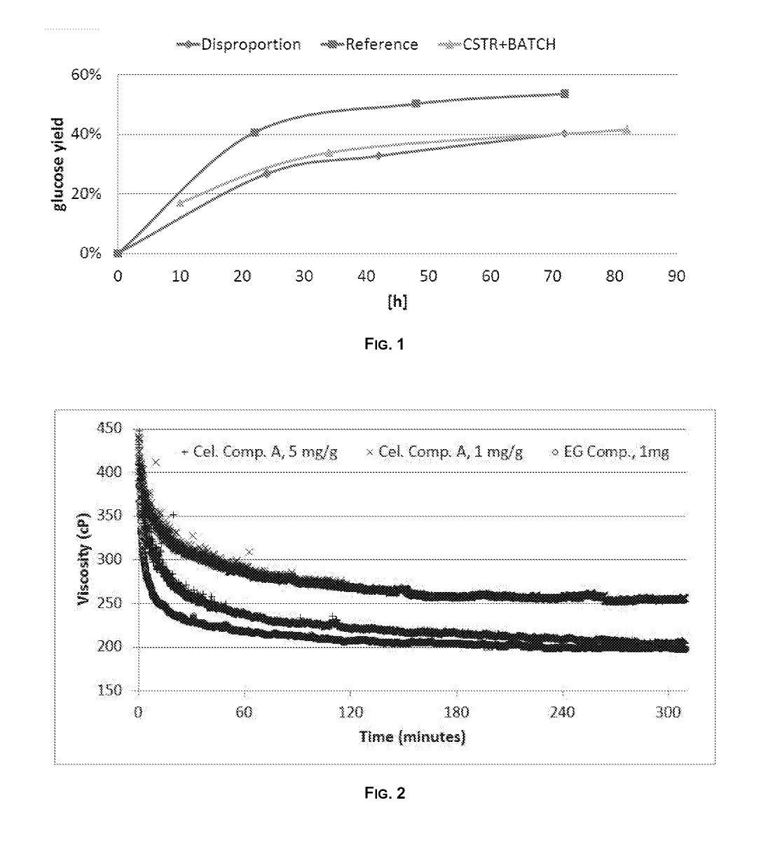

Example 1 provides a two stage hydrolysis of steam exploded wheat straw where a first stage is conducted in CSTR and a second stage is conducted in batch reactor, as compared to the hydrolysis of steam exploded wheat straw in a pure batch process. It is shown in FIG. 1 that a gap in yield is observed, where the process with a first stage CSTR presents a lower glucose yield than the yield from a pure batch process.

In order to improve the hydrolysis yield, particularly in processes comprising a continuous reactor, the present inventors have discovered that improved yields can be achieved through administration of enzymes as at least two separate enzyme compositions in a multi-stage hydrolysis. Such improvement is achieved while keeping enzyme loading low. As described herein, hydrolysis of cellulosic material is performed enzymatically by two or more enzyme compositions in two or more stages of hydrolysis. In an embodiment the invention provides processes including multi-stage hydrolysis including a first stage of hydrolysis comprising adding enzymes to reduce inhibitors and/or reduce viscosity and a second stage of hydrolysis comprising adding hydrolyzing enzymes. In an embodiment, such process is sufficient to improve or increase sugar yields in a hydrolysis process comprising use of a continuous reactor, as compared to yields from a process that does not use a multi-stage enzyme administration. In another embodiment the yields from a hydrolysis process comprising use of a continuous reactor can be improved to a level similar to the yields obtained from a pure batch process. In a particular embodiment the improvement or increase in sugar yield from a process of the invention is sufficient to decrease or eliminate an observed gap in yield between a single-stage hydrolysis process including a continuous reactor, as compared to a pure batch process, where the process including a continuous reactor has a lower sugar yield than the pure batch process.

In an embodiment, processes of the invention include a first stage of hydrolysis where the enzyme activity is sufficient to reduce the inhibitors, e.g., xylo-oligomers, and/or to reduce the viscosity as compared to the inhibitors present in and the viscosity of a pretreated lignocellulosic material not subjected to such a first stage of hydrolysis.

The present invention therefore relates to processes of improving a glucose or xylose yield of saccharification of a lignocellulosic material in a continuous reactor, the process comprising the steps of: a first stage comprising saccharifying a lignocellulosic material in a continuous reactor with a first enzyme composition comprising a xylanase, a beta-xylosidase and an endoglucanase; and a second stage comprising continuing saccharification of the lignocellulosic material, comprising combining the material of step a) with a second enzyme composition comprising one or more cellulases to form a hydrolyzate, wherein the hydrolyzate has a glucose yield or a xylose yield that is improved as compared to the yield from a process comprising a single saccharification step. In one embodiment the continuous reactor is a CSTR. In another embodiment, the processes further comprise recovering the hydrolyzate. Soluble products of degradation of the cellulosic material can be separated from insoluble cellulosic material using a method known in the art such as, for example, centrifugation, filtration, or gravity settling. In an embodiment the first enzyme composition is added in a first stage of hydrolysis and the second enzyme composition is added in a later (e.g., second) stage of hydrolysis. In a further embodiment, the stages of hydrolysis are conducted at a pH independently selected from about 3.5 to about 5.5. In a still further embodiment, the first stage of hydrolysis is conducted at a lower pH than the second stage of hydrolysis. In another embodiment, the second enzyme composition is added at least about 2 hours, at least about 3 hours, at least about 5 hours, at least about 10 hours, or at least about 20 hours following contacting of the lignocellulosic material and the first enzyme composition. In a particular embodiment the glucose yield or xylose yield is increased to a level similar to a yield obtained from a pure batch saccharification process.

The present invention further relates to processes of multi-stage hydrolysis of a lignocellulosic material, the process comprising the steps of a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material, and an endoglucanase in an amount of about 2.84 U to about 117.2 U per gram of the lignocellulosic material to form a first saccharified material, and a second stage comprising continuing saccharification of the lignocellulosic material, comprising combining the first saccharified material with a second enzyme composition comprising one or more cellulases. In an embodiment the first enzyme composition is added in a first stage of hydrolysis and the second enzyme composition is added in a later (e.g., second) stage of hydrolysis. In a further embodiment, the stages of hydrolysis are conducted at a pH independently selected from about 3.5 to about 5.5. In a still further embodiment, the first stage of hydrolysis is conducted at a lower pH than the second stage of hydrolysis. In another embodiment, the second enzyme composition is added at least about 2 hours, at least about 3 hours, at least about 5 hours, at least about 10 hours, or at least about 20 hours following contacting of the lignocellulosic material and the first enzyme composition. In another embodiment the saccharification comprising combining the lignocellulosic material with a first enzyme composition is performed in a continuous reactor. In a further embodiment the continuous reactor is a CSTR.

In a still further embodiment, the first stage of hydrolysis is conducted at a lower pH than the second stage of hydrolysis. In another embodiment, the second enzyme composition is added at least about 2 hours, at least about 3 hours, at least about 5 hours, at least about 10 hours, or at least about 20 hours following contacting of the lignocellulosic material and the first enzyme composition.

Enzymatic hydrolysis (i.e., saccharification) is preferably carried out in a suitable aqueous environment under conditions that may be readily determined by one skilled in the art. In one embodiment, hydrolysis is performed under conditions suitable for the activity of the enzyme composition, preferably optimal for the enzyme composition.

The hydrolysis is generally performed in stirred-tank reactors or fermentors under controlled pH, temperature, and mixing conditions. Suitable process time, temperature and pH conditions may readily be determined by one skilled in the art.

As used herein “multi-stage hydrolysis” or “multi-stage saccharification” refers to a hydrolysis performed in two or more stages. Stages of hydrolysis may include, but are not limited to, use of one or more reactors, variations in temperature during the hydrolysis process, variations in pH during the hydrolysis process, variations in mixing or stirring, variations in timing (e.g., length of time of each stage) during the hydrolysis process, and variations of enzyme addition during the hydrolysis process. In various embodiments of the invention, stages of hydrolysis may comprise one or more of: different reactors, different temperatures, different pH, different mixing/stirring, and administration of different enzyme compositions.

For example, the hydrolysis may last up to 200 hours, but is typically performed for preferably about 12 to about 120 hours, e.g., about 16 to about 72 hours or about 24 to about 48 hours. In an embodiment of the invention a first stage of hydrolysis is carried out for about 3 to about 36 hours, e.g., about 15 to about 30 hours. In another embodiment of the invention a second stage of hydrolysis is carried out for about for about 3 to about 36 hours, e.g., about 15 to about 30 hours.

The hydrolysis temperature is in the range of preferably about 25° C. to about 70° C., e.g., about 30° C. to about 65° C., about 40° C. to about 60° C., or about 50° C. to about 55° C. In one embodiment the first stage of hydrolysis and the second stage of hydrolysis are performed at about the same temperature. In another embodiment a first stage of hydrolysis has a temperature that is varied from the temperature of a second stage of hydrolysis.

The pH of hydrolysis is in the range of preferably about 3 to about 8, e.g., about 3.5 to about 7, about 4 to about 6, or about 4.5 to about 5.5. In an embodiment of the invention the pH of a first stage and a second stage are independently selected from a pH of about 3.5 to about 5.5, i.e., about 3.5, about 3.6, about 3.7, about 3.8, bout 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, or about 5.5. In an embodiment a first stage of hydrolysis has a pH that is varied from the pH of a second stage of hydrolysis. In another embodiment a first stage of hydrolysis has a pH that is lower than the pH of a second stage of hydrolysis.

The dry solids content is in the range of preferably about 5 to about 50 wt. %, e.g., about 10 to about 40 wt. % or about 15 to about 30 wt. %.

In a preferred embodiment, the invention provides processes comprising a multi-stage hydrolysis in which a different enzyme composition is provided in each stage. In an embodiment the hydrolysis is conducted in more than one reactor in series. In a further embodiment at least two stages are conducted at different pH

### Enzymes for Hydrolysis

The present invention relates to use of enzymes in a multi-stage hydrolysis comprising administration of the enzymes as two or more enzyme compositions. In a particular embodiment the invention comprises administration of different enzyme compositions in a multi-stage hydrolysis process. Preferably, a first enzyme composition is sufficient to reduce inhibitors or reduce viscosity in a substrate-containing slurry. In a further embodiment the first enzyme composition is sufficient to both reduce inhibitors and reduce viscosity in a substrate-containing slurry. In an embodiment a first enzyme composition of the invention comprises a xylanase, a beta-xylosidase and a cellulase. In a preferred embodiment the cellulase is an endoglucanase. In a further embodiment a first enzyme composition comprises at least endoglucanase. A multi-stage hydrolysis process of the invention may further comprise one or more additional enzyme compositions. In an embodiment, the process further comprises administration of a second enzyme composition comprising one or more cellulases.

One or more (e.g., several) components of the enzyme compositions may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme compositions. It is understood herein that the recombinant proteins may be heterologous (e.g., foreign) and/or native to the host cell. One or more (e.g., several) components of the enzyme compositions may be produced as monocomponents, which are then combined to form the enzyme compositions. The enzyme compositions may be a combination of multicomponent and monocomponent protein preparations. The compositions may be further combined with one or more additional enzyme compositions.

The enzymes used in processes of the present invention may be in any form suitable for use, such as, for example, a fermentation broth formulation or a cell composition, a cell lysate with or without cellular debris, a semi-purified or purified enzyme composition, or a host cell as a source of the enzymes. The enzyme compositions may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

The optimum amounts of the enzymes and polypeptides depend on several factors including, but not limited to, the mixture of cellulolytic enzymes and/or hemicellulolytic enzymes, the cellulosic material, the concentration of cellulosic material, the pretreatment of the cellulosic material, temperature, time, pH, and inclusion of a fermenting organism (e.g., for Simultaneous Saccharification and Fermentation).

In one embodiment a first enzyme composition of the invention comprises a xylanase, a beta-xylosidase and a cellulase. In a preferred embodiment the cellulase is an endoglucanase. In a particular embodiment of the invention, the invention provides a multi-stage hydrolysis process, wherein a first enzyme composition is added in a first stage of hydrolysis. In a further embodiment of the invention, the invention provides a multi-stage hydrolysis process, wherein a second enzyme composition is added in a second or subsequent stage of hydrolysis, following administration of a first enzyme composition in a prior stage of hydrolysis. In a particular embodiment a first enzyme composition is administered in a first stage of hydrolysis in a continuous reactor.

The first enzyme composition is present as about 1% to about 99%, e.g., about 1% to about 45%, about 2% to about 40%, or about 5% to about 35% of the total enzyme protein added during hydrolysis.

The second enzyme composition is present as about 1% to about 99%, e.g., about 55% to about 99%, about 60% to about 98%, or about 65% to about 95% of the total enzyme protein added during hydrolysis. In a preferred embodiment, the combined first enzyme composition and second enzyme composition comprise 100% of the total enzyme protein added during hydrolysis. In another embodiment, the ratio of enzyme protein of a first enzyme composition to enzyme protein of a second enzyme composition by weight is about 1:2.

In one embodiment, an effective amount of total enzyme protein added during hydrolysis to the cellulosic material is about 0.5 to about 15 mg, e.g., about 0.5 to about 10 mg, about 0.5 to about 9 mg, about 0.5 to about 6 mg, or about 0.5 to about 5 mg per g of the cellulosic material. In a preferred embodiment the total enzyme protein added during hydrolysis comprising all enzyme compositions added in all stages of hydrolysis is about 4 to about 10 mg, or about 4 to about 7 mg per g of the cellulosic material.

The enzymes may be present or added during hydrolysis (i.e., saccharification) in amounts effective from about 0.001 to about 5.0 wt % of solids (TS), more preferably from about 0.025 to about 4.0 wt % of solids, and most preferably from about 0.005 to about 2.0 wt % of solids (TS).

The enzymes in enzyme compositions of the invention may be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained by, e.g., site-directed mutagenesis or shuffling.

Each polypeptide may be a bacterial polypeptide. For example, each polypeptide may be a Gram-positive bacterial polypeptide having enzyme activity, or a Gram-negative bacterial polypeptide having enzyme activity.

Each polypeptide may also be a fungal polypeptide, e.g., a yeast polypeptide or a filamentous fungal polypeptide.

Chemically modified or protein engineered mutants of polypeptides may also be used.

One or more (e.g., several) components of the enzyme compositions may be a recombinant component, i.e., produced by cloning of a DNA sequence encoding the single component and subsequent cell transformed with the DNA sequence and expressed in a host (see, for example, WO 91/17243 and WO 91/17244). The host may be a heterologous host (enzyme is foreign to host), but the host may under certain conditions also be a homologous host (enzyme is native to host). Monocomponent cellulolytic proteins may also be prepared by purifying such a protein from a fermentation broth.

In a particular embodiment a first enzyme composition comprises a xylanase, a beta-xylosidase and a cellulase. In a preferred embodiment the cellulase is an endoglucanase. In a preferred embodiment the first enzyme composition has endo-acting and exo-acting activity in hydrolysis of xylans. In a further embodment the first enzyme composition has activity in viscosity reduction.

Examples of xylanases useful in the processes of the present invention include, but are not limited to, xylanases from Aspergillus aculeatus (GENSEQP™ Accession No. AAR63790; WO 94/21785), Aspergillus fumigatus (WO 2006/078256), Penicillium pinophilum (WO 2011/041405), Penicillium sp. (WO 2010/126772), Talaromyces lanuginosus GH11 (WO 2012/130965), Talaromyces leycettanus GH10 (GENSEQP™ Accession No. BAK46118), Talaromyces thermophilus GH11 (WO 2012/130950), Thielavia terrestris NRRL 8126 (WO 2009/079210), and Trichophaea saccata GH10 (WO 2011/057083).

In one embodiment the xylanase is selected from the group consisting of: (i) a xylanase comprising or consisting of the mature polypeptide of SEQ ID NO: 12; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 12; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 11; and (iv) a xylanase encoded by a polynucleotide that hybridizes under at least high stringency conditions, e.g., very high stringency conditions, with the mature polypeptide coding sequence of SEQ ID NO: 11 or the full-length complement thereof.

In another embodiment the xylanase is selected from the group consisting of: (i) a xylanase comprising or consisting of the mature polypeptide of SEQ ID NO: 14; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 14; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 13; and (iv) a xylanase encoded by a polynucleotide that hybridizes under at least high stringency conditions, e.g., very high stringency conditions, with the mature polypeptide coding sequence of SEQ ID NO: 13 or the full-length complement thereof;

In another embodiment the xylanase is selected from the group consisting of: (i) a xylanase comprising or consisting of the mature polypeptide of SEQ ID NO: 22; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 22; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 21; and (iv) a xylanase encoded by a polynucleotide that hybridizes under at least high stringency conditions, e.g., very high stringency conditions, with the mature polypeptide coding sequence of SEQ ID NO: 21 or the full-length complement thereof.

Examples of beta-xylosidases useful in the processes of the present invention include, but are not limited to, beta-xylosidases from Aspergillus fumigatus (GENSEQP™ Accession No. AZ105042; WO 2013/028928), Neurospora crassa (SwissProt:Q7SOW4), Talaromyces emersonii (SwissProt:Q8X212), Trichoderma reesei (UniProtKB/TrEMBL:Q92458), and Trichoderma reesei such as the mature polypeptide of GENSEQP™ Accession No. AZI04896.

In one embodiment the beta-xylosidase is selected from the group consisting of: (i) a beta-xylosidase comprising or consisting of the mature polypeptide of SEQ ID NO: 16; (ii) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 16; (iii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 15; and (iv) a beta-xylosidase encoded by a polynucleotide that hybridizes under at least high stringency conditions, e.g., very high stringency conditions, with the mature polypeptide coding sequence of SEQ ID NO: 15 or the full-length complement thereof.

In another embodiment the beta-xylosidase is selected from the group consisting of: (i) a beta-xylosidase comprising or consisting of the mature polypeptide of SEQ ID NO: 24; (ii) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 24; (iii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 23; and (iv) a beta-xylosidase encoded by a polynucleotide that hybridizes under at least high stringency conditions, e.g., very high stringency conditions, with the mature polypeptide coding sequence of SEQ ID NO: 23 or the full-length complement thereof.

Examples of bacterial endoglucanases that may be used in the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that may be used in the present invention, include, but are not limited to, Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Humicola insolens endoglucanase V, Melanocarpus albomyces endoglucanase (GenBank:MAL515703), Myceliophthora thermophila CBS 117.65 endoglucanase, Neurospora crassa endoglucanase (GenBank:XM_324477), Thermoascus aurantiacus endoglucanase I (GenBank:AF487830), Thermoascus aurantiacus Cel5 endoglucanase II (WO2011/057140), Trichoderma reesei endoglucanase I (Penttila et al., 1986, Gene 45: 253-263), Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), and Trichoderma reesei strain No. VTT-D-80133 endoglucanase (GenBank:M15665).

In another embodiment, an enzyme composition of the invention further or even further comprises a Trichoderma endoglucanase I or a homolog thereof. In another aspect, an enzyme composition further comprises a Trichoderma reesei endoglucanase I or a homolog thereof. In another aspect, an enzyme composition further comprises a Trichoderma reesei Cel7B endoglucanase I (GENBANK™ accession no. M15665) or a homolog thereof. In another aspect, the Trichoderma reesei endoglucanase I or a homolog thereof is native to the host cell.

In another aspect, an enzyme composition of the invention further or even further comprises a Trichoderma endoglucanase II or a homolog thereof. In another aspect, an enzyme composition further comprises a Trichoderma reesei endoglucanase II or a homolog thereof. In another aspect, an enzyme composition further comprises a Trichoderma reesei Cel5A endoglucanase II (GENBANK™ accession no. M19373) or a homolog thereof. In another aspect, the Trichoderma reesei endoglucanase II or a homolog thereof is native to the host cell.

In one embodiment the endoglucanase is selected from the group consisting of: (i) an endoglucanase comprising or consisting of the mature polypeptide of SEQ ID NO: 20; (ii) an endoglucanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 20; (iii) an endoglucanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 19; and (iv) an endoglucanase encoded by a polynucleotide that hybridizes under at least high stringency conditions, e.g., very high stringency conditions, with the mature polypeptide coding sequence of SEQ ID NO: 19 or the full-length complement thereof.

A first enzyme composition of the invention comprises a cellulase sufficient to reduce viscosity of the substrate-containing slurry. In a preferred embodiment endoglucanase is present in a first enzyme composition as about 0.5 to about 100% of the total enzyme protein added during hydrolysis, e.g., about 1% to about 90%, about 5% to about 50%, about 7% to about 25%, or about 7% to about 11%. In one embodiment endoglucanase is about 30% to about 50% of the enzyme protein in the first enzyme composition.

The amount of cellulase in the first enzyme composition may be determined as described in Example 8 and measured in U/mg total enzyme. Therefore, in an embodiment, the effective amount of cellulase in a first enzyme composition of the present invention is about 2.84 U to about 117.2 U, e.g., about 3.9 U to about 117.2 U, about 31.2 to about 91.3, about 3.9 U to about 78.1 U, about 3.9 U to about 70.3 U, about 3.9 U to about 46.9 U, or about 3.9 U to about 39.2 U per g of the cellulosic material.

In an embodiment, the amount of xylanase in a first enzyme composition of the present invention is 0% to 30% of the total enzyme protein added during hydrolysis, e.g., 0.5% to 30%, 1.0% to 27.5%, 1.5% to 25%, 2% to 22.5%, 2.5% to 20%, 3% to 19%, 3.5% to 18%, and 4% to 17% of the total enzyme protein added during hydrolysis.

The amount of xylanase may be determined as described in Example 9 and measured in U/mg total enzyme. Therefore in an embodiment, the effective amount of xylanase in a first enzyme composition of the present invention is about 4.3 U to about 716.1 U, e.g., about 23.8 U to about 716.1 U, about 23.8 U to about 477.4 U, about 190.9 U to about 477.4 U, about 23.8 U to about 429.7 U, about 23.8 U to about 286.4 U, or about 23.8 U to about 238.7 U per g of the cellulosic material.

In another embodiment, the amount of beta-xylosidase in a first enzyme composition of the present invention is 0% to 50% of the total enzyme protein added during hydrolysis, e.g., 0.5% to 30%, 1.0% to 27.5%, 1.5% to 25%, 2% to 22.5%, 2.5% to 22%, 3% to 19%, 3.5% to 18%, and 4% to 17% of the total enzyme protein added during hydrolysis.

The amount of beta-xylosidase may be determined as described in Example 10 and measured in U/mg total enzyme. Therefore in an embodiment, the effective amount of beta-xylosidase in a first enzyme composition of the present invention is about 0.005 U to about 0.86 U, e.g., about 0.03 U to about 0.86 U, about 0.03 U to about 0.57 U, about 0.23 U to about 0.57 U, about 0.03 U to about 0.51 U, about 0.03 U to about 0.34 U, or about 0.03 U to about 0.29 U per g of the cellulosic material.

In still another embodiment, the amount of xylanase and beta-xylosidase in a first enzyme composition of the present invention, taken together, is about 0.5 to about 100% of the total enzyme protein added during hydrolysis, e.g., about 1% to about 90%, about 5% to about 50%, about 15% to about 25%, about 20% to about 23%. In one embodiment the xylanase and beta-xylosidase in a first enzyme composition of the present invention, taken together, is about 40% to about 70% of the enzyme protein in the first enzyme composition.

In a particular embodiment a first enzyme composition is derived from Trichoderma reesei, further comprising a xylanase of SEQ ID NO: 12, 14 or 22, a beta xylosidase of SEQ ID NO: 16 or 24, and an endoglucanase of SEQ ID NO: 20.

In an embodiment the first enzyme composition is or comprises a commercial hemicellulolytic enzyme composition. Examples of commercial hemicellulolytic enzyme compositions suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Danisco US Inc.), ACCELLERASE® XY (Danisco US Inc.), ACCELLERASE® XC (Danisco US Inc.), ACCELLERASE® TRIO (Danisco US Inc.), ECOPULP® TX-200A (Roal Oy LLC), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

In a further embodiment a second enzyme composition comprises a cellulolytic enzyme composition comprising one or more (e.g., several) cellulolytic enzymes. In another embodiment, the enzymes in the second enzyme composition comprise or further comprise one or more (e.g., several) hemicellulolytic enzymes. In another embodiment, the enzymes in the second enzyme composition comprise one or more (e.g., several) cellulolytic enzymes and one or more (e.g., several) hemicellulolytic enzymes. In another embodiment, the enzymes in the second enzyme composition comprise one or more (e.g., several) enzymes selected from the group of cellulolytic enzymes and hemicellulolytic enzymes. In another embodiment, the enzymes in the second enzyme composition comprise a cellobiohydrolase. In a further embodiment the cellobiohydrolase is a cellobiohydrolase I, a cellobiohydrolase II, or a combination of a cellobiohydrolase I and a cellobiohydrolase II. In another embodiment, the enzymes in the second enzyme composition comprise a beta-glucosidase. In another embodiment, the enzymes in the second enzyme composition comprise an AA9 polypeptide. In another embodiment, the enzymes in the the second enzyme composition comprise an endoglucanase. In still another embodiment the enzymes in the second enzyme composition comprise a xylanase. In a still further embodiment the enzymes in the second enzyme composition comprise a beta-xylosidase.

In a further embodiment the second enzyme composition comprises enzymes selected from the group consisting of a cellobiohydrolase, a beta glucosidase and an AA9 polypeptide having cellulolytic enhancing activity.

Examples of cellobiohydrolases useful in the present invention include, but are not limited to, Aspergillus aculeatus cellobiohydrolase II (WO 2011/059740), Aspergillus fumigatus cellobiohydrolase I (GENSEQP™ Accession No. AZI04842), Aspergillus fumigatus cellobiohydrolase II (GENSEQP™ Accession No. AZI04854), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank:AY690482), Talaromyces emersonii cellobiohydrolase I (GenBank:AF439936), Talaromyces leycettanus cellobiohydrolase I (GENSEQP™ Accession No. AZY49536), Talaromyces leycettanus cellobiohydrolase II (GENSEQP™ Accession No. AZY49446), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (GENSEQP™ Accession No. AEA33202), an Aspergillus fumigatus variant such as GENSEQP™ Accession No. AZU67153, Aspergillus niger (Dan et al., 2000, J. Biol. Chem. 275: 4973-4980), Aspergillus oryzae (WO 02/095014) or the fusion protein having beta-glucosidase activity disclosed in WO 2008/057637, Penicillium brasilianum IBT 20888 (WO 2007/019442 and WO 2010/088387), Thielavia terrestris (WO 2011/035029), Trichophaea saccata (WO 2007/019442) and Trichoderma reesei.

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

In the processes of the present invention, any AA9 polypeptide may be used as a component of an enzyme composition.

Examples of AA9 polypeptides useful in the processes of the present invention include, but are not limited to, AA9 polypeptides from Aspergillus aculeatus (WO 2012/125925), Aspergillus fumigatus (WO 2010/138754), Aurantiporus alborubescens (WO 2012/122477), Chaetomium thermophilum (WO 2012/101206), Humicola insolens (WO 2012/146171), Malbranchea cinnamomea (WO 2012/101206), Myceliophthora thermophila (WO 2009/085935, WO 2009/085859, WO 2009/085864, WO 2009/085868, WO 2009/033071, WO 2012/027374, and WO 2012/068236), Penicillium pinophilum (WO 2011/005867), Penicillium thomii (WO 2012/122477), Penicillium sp. (emersonii) (WO 2011/041397 and WO 2012/000892), Talaromyces emersonii (WO 2012/000892), Talaromyces leycettanus (WO 2012/101206), Talaromyces stipitatus (WO 2012/135659), Talaromyces thermophilus (WO 2012/129697 and WO 2012/130950), Thermoascus aurantiacus (WO 2005/074656 and WO 2010/065830), Thermoascus crustaceous (WO 2011/041504), Thermoascus sp. (WO 2011/039319), Thermomyces lanuginosus (WO 2012/113340, WO 2012/129699, WO 2012/130964, and WO 2012/129699), Thielavia terrestris (WO 2005/074647, WO 2008/148131, and WO 2011/035027), Trametes versicolor (WO 2012/092676 and WO 2012/093149), Trichoderma reesei (WO 2007/089290 and WO 2012/149344), and Trichophaea saccata (WO 2012/122477).

In one embodiment, the AA9 polypeptide is used in the presence of a soluble activating divalent metal cation according to WO 2008/151043, e.g., copper.

In another embodiment, the AA9 polypeptide is used in the presence of a dioxy compound, a bicylic compound, a heterocyclic compound, a nitrogen-containing compound, a quinone compound, a sulfur-containing compound, or a liquor obtained from a pretreated cellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

In one embodiment, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another embodiment, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide may be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme composition. The liquor may be separated from the treated material using a method standard in the art, such as filtration, sedimentation, or centrifugation.

In one embodiment, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In an embodiment, a second enzyme composition comprises a cellobiohydrolase I, a cellobiohydrolase II, a beta-glucosidase or variant thereof, and an AA9 polypeptide. In a particular embodiment a second enzyme composition is derived from Trichoderma reesei, further comprising AA9 (GH61) polypeptide having cellulolytic enhancing activity set forth as SEQ ID NO: 2 in WO 2011/041397, a beta-glucosidase (SEQ ID NO: 2 in WO 2005/047499) variant (F100D, S283G, N456E, F512Y) set forth in WO 2012/044915; a CBH I set forth as SEQ ID NO: 6 in WO 2011/057140 and a CBH II set forth as SEQ ID NO: 18 in WO 2011/057140.

In a further embodiment a second enzyme composition comprises one or more hemicellulases. In an embodiment, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

Examples of xylanases and xylosidases are as set forth herein with respect to the first enzyme composition.

Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Examples of feruloyl esterases (ferulic acid esterases) useful in the processes of the present invention include, but are not limited to, feruloyl esterases from Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases useful in the processes of the present invention include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (Swiss Prot:Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

In a still further embodiment a second enzyme composition comprises one or more oxidoreductases. Examples of oxidoreductases useful in the processes of the present invention include, but are not limited to, Aspergillus fumigatus catalase, Aspergillus lentilus catalase, Aspergillus niger catalase, Aspergillus oryzae catalase, Humicola insolens catalase, Neurospora crassa catalase, Penicillium emersonii catalase, Scytalidium thermophilum catalase, Talaromyces stipitatus catalase, Thermoascus aurantiacus catalase, Coprinus cinereus laccase, Myceliophthora thermophila laccase, Polyporus pinsitus laccase, Pycnoporus cinnabarinus laccase, Rhizoctonia solani laccase, Streptomyces coelicolor laccase, Coprinus cinereus peroxidase, Soy peroxidase, Royal palm peroxidase.

In an embodiment the second enzyme composition is or comprises a commercial cellulolytic enzyme preparation. Examples of commercial cellulolytic enzyme preparations suitable for use in the present invention include, for example, CELLIC® CTec (Novozymes A/S), CELLIC® CTec2 (Novozymes A/S), CELLIC® Ctec3 (Novozymes A/S), CELLUCLAST® (Novozymes A/S), CELLUZYME™ (Novozymes A/S), CEREFLO® (Novo Nordisk A/S), and ULTRAFLO® (Novozymes A/S), ACCELLERASE® (Danisco US Inc.), LAMINEX® (Danisco US Inc.), SPEZYME® CP (Danisco US Inc.), ROHAMENT® 7069 W (AB Enzymes), FIBREZYME® LDI (Dyadic International, Inc.), FIBREZYME® LBR (Dyadic International, Inc.), or VISCOSTAR™ 150L (Dyadic International, Inc.).

In one embodiment, the amount of cellobiohydrolase I in a second enzyme composition of the present invention is 5% to 60% of the total enzyme protein added during hydrolysis, e.g., 7.5% to 55%, 10% to 50%, 12.5% to 45%, 15% to 40%, 17.5% to 35%, and 20% to 30% of the total enzyme protein added during hydrolysis.

In another embodiment, the amount of cellobiohydrolase II in a second enzyme composition of the present invention is 2.0-40% of the total enzyme protein added during hydrolysis, e.g., 3.0% to 35%, 4.0% to 30%, 5% to 25%, 6% to 20%, 7% to 15%, and 7.5% to 12% of the total enzyme protein added during hydrolysis.

In another embodiment, the amount of beta-glucosidase in a second enzyme composition of the present invention is 0% to 30% of the total enzyme protein added during hydrolysis, e.g., 1% to 27.5%, 1.5% to 25%, 2% to 22.5%, 3% to 20%, 4% to 19%, % 4.5 to 18%, 5% to 17%, and 6% to 16% of the total enzyme protein added during hydrolysis.

In another embodiment, the amount of AA9 polypeptide in a second enzyme composition of the present invention is 0% to 50% of the total enzyme protein added during hydrolysis, e.g., 2.5% to 45%, 5% to 40%, 7.5% to 35%, 10% to 30%, 12.5% to 25%, and 15% to 25% of the total enzyme protein added during hydrolysis.

In one embodiment the components of the first enzyme composition are mixed or blended prior to addition to the reactor. In another embodiment, the components of the second enzyme composition are mixed or blended prior to addition to the reactor. In another embodiment the first enzyme composition and the second enzyme composition are added in different stages of hydrolysis in a multi-stage hydrolysis process. In a further embodiment, the first enzyme composition, or component parts thereof, is added to a reactor before, concurrent with, or after addition of the lignocellulosic material to the reactor. In a still further embodiment, the second enzyme composition, or component parts thereof, is added to a reactor after addition of the lignocellulosic material and the first enzyme composition to the reactor.

One or more (e.g., several) of the enzymes added during hydrolysis may be wild-type proteins expressed by the host strain, recombinant proteins, or a combination of wild-type proteins expressed by the host strain and recombinant proteins. For example, one or more (e.g., several) enzymes may be native proteins of a cell, which is used as a host cell to express recombinantly the enzymes added during hydrolysis.

The enzyme compositions may be prepared in accordance with methods known in the art and may be in the form of a liquid or a dry composition. The compositions may be stabilized in accordance with methods known in the art.

The enzyme compositions may result from a single fermentation or may be a blend of two or more fermentations, e.g., three, four, five, six, seven, etc. fermentations.

The enzyme compositions may be in any form suitable for use, such as, for example, a crude fermentation broth with or without cells removed, a cell lysate with or without cellular debris, a semi-purified or purified enzyme composition, or a Trichoderma host cell as a source of the enzymes. The enzyme compositions may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

The enzyme compositions may also be a fermentation broth formulation or a cell composition. The fermentation broth product further comprises additional ingredients used in the fermentation process, such as, for example, cells (including, the host cells containing the gene encoding the polypeptide of the present invention which are used to produce the polypeptide), cell debris, biomass, fermentation media and/or fermentation products. In some embodiments, the composition is a cell-killed whole broth containing organic acid(s), killed cells and/or cell debris, and culture medium.

The term “fermentation broth” refers to a composition produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains live cells, killed cells and/or cell debris. In one embodiment, the composition comprises live cells. In another embodiment, killed cells, and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid slurry, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by the methods described in WO 90/15861 or WO 2010/096673.

The fermentation may be any method of cultivation of a cell resulting in the expression or isolation of an enzyme or protein. Fermentation may, therefore, be understood as comprising shake flask cultivation, or small-scale or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

### Fermentation

The fermentable sugars obtained from the hydrolyzed cellulosic material may be fermented by one or more (e.g., several) fermenting microorganisms capable of fermenting the sugars directly or indirectly into a desired fermentation product.

“Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and may easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation may be separate or simultaneous. Hydrolysis as described herein includes multi-stage hydrolysis. Where hydrolysis and fermentation are simultaneous, fermentation is carried out with one or more stages of hydrolysis.

Hydrolysis (saccharification) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the cellulosic material to fermentable sugars, e.g., glucose, cellobiose, and pentose monomers, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis (saccharification), fermentation, or a combination thereof, can be used in the practicing processes of the present invention.

Still further, the invention relates to processes of producing a fermentation product from a lignocellulosic material, the process comprising the steps of contacting the lignocellulosic material with 1) a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U per gram of the lignocellulosic material and 2) a second enzyme composition comprising one or more cellulases to form a hydrolyzate, and fermenting the hydrolyzate to produce a fermentation product. In an embodiment the first enzyme composition is added in a first stage of hydrolysis and the second enzyme composition is added in a later (e.g., second) stage of hydrolysis. In a further embodiment, the stages of hydrolysis are conducted at a pH independently selected from about 3.5 to about 5.5. In a still further embodiment, the first stage of hydrolysis is conducted at a lower pH than the second stage of hydrolysis. In another embodiment, the second enzyme composition is added at least about 2 hours, at least about 3 hours, at least about 5 hours, at least about 10 hours, or at least about 20 hours following contacting of the lignocellulosic material and the first enzyme composition. In another embodiment the saccharification comprising combining the lignocellulosic material with a first enzyme composition is performed in a continuous reactor. In a further embodiment the continuous reactor is a CSTR.

The present invention also relates to processes of fermenting a lignocellulosic material, comprising: fermenting the lignocellulosic material with one or more (e.g., several) fermenting microorganisms, wherein the lignocellulosic material is hydrolyzed with 1) a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U per gram of the lignocellulosic material and 2) a second enzyme composition comprising one or more cellulases to form a hydrolyzate. In one embodiment, the fermenting of the cellulosic material produces a fermentation product. In another embodiment, the processes further comprise recovering the fermentation product from the fermentation.

Any suitable hydrolyzed cellulosic material may be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is (are) added, such as, a medium resulting from a saccharification process, as well as a medium used in a simultaneous saccharification and fermentation process (SSF).

Suitable fermenting organisms used according of processes of the invention are described below in the “Fermenting Organism”-section below

### Fermenting Organism

“Fermenting organism” or “fermenting microorganism” refers to any microorganism, including bacterial and fungal organisms, suitable for use in a desired fermentation process to produce a fermentation product. The fermenting organism may be hexose C₆) and/or pentose (C₅) fermenting organisms, or a combination thereof. Both hexose and pentose fermenting organisms are well known in the art. Suitable fermenting organisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment C₆ sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae. Preferred yeast includes strains of the Saccharomyces spp., preferably Saccharomyces cerevisiae.

Examples of fermenting organisms that can ferment C₅ sugars include bacterial and fungal organisms, such as yeast. Preferred C₅ fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis, or Candida utilis. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, 1996, supra).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

Commercially available yeast suitable for ethanol production include, e.g., BIO-FERM® AFT and XR, ETHANOL RED® yeast, FALI®, FERMIOL®, GERT STRAND™ (Gert Strand AB, Sweden), SUPERSTART™ and THERMOSACC® fresh yeast.

In an embodiment, the fermenting organism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (cofermentation) (Chen and Ho, 1993, Appl. Biochem. Biotechnol. 39-40: 135-147; Ho et al., 1998, Appl. Environ. Microbiol. 64: 1852-1859; Kotter and Ciriacy, 1993, Appl. Microbiol. Biotechnol. 38: 776-783; Walfridsson et al., 1995, Appl. Environ. Microbiol. 61: 4184-4190; Kuyper et al., 2004, FEMS Yeast Research 4: 655-664; Beall et al., 1991, Biotech. Bioeng. 38: 296-303; Ingram et al., 1998, Biotechnol. Bioeng. 58: 204-214; Zhang et al., 1995, Science 267: 240-243; Deanda et al., 1996, Appl. Environ. Microbiol. 62: 4465-4470; WO 03/062430).

It is well known in the art that the organisms described above may also be used to produce other substances, as described herein.

The fermenting organism is typically added to the degraded cellulosic material or hydrolyzate and the fermentation is performed for about 8 to about 96 hours, such as about 24 to about 60 hours. The temperature is typically between about 26° C. to about 60° C., in particular about 32° C. or 50° C., and at about pH 3 to about pH 8, such as around pH 4-5, 6, or 7.

In one embodiment, the yeast and/or another microorganism are applied to the degraded cellulosic material and the fermentation is performed for about 12 to about 96 hours, such as typically 24-60 hours. In another embodiment, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation may be found in, e.g., “The Alcohol Textbook” (Editors K. Jacques, T. P. Lyons and D. R. Kelsall, Nottingham University Press, United Kingdom 1999), which is hereby incorporated by reference.

For ethanol production, following the fermentation the fermented slurry may be distilled to extract the ethanol. The ethanol obtained according to processes of the invention may be used as, e.g., fuel ethanol, drinking ethanol, i.e., potable neutral spirits, or industrial ethanol.

### Fermentation Stimulators

A fermentation stimulator may be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

### Fermentation Products

Processes of the present invention can be used to saccharify the lignocellulosic material to fermentable sugars and to convert the fermentable sugars to many useful fermentation products, e.g., fuel (ethanol, n-butanol, isobutanol, biodiesel, jet fuel) and/or platform chemicals (e.g., acids, alcohols, ketones, gases, oils, and the like). The production of a desired fermentation product from the cellulosic material typically involves pretreatment, enzymatic hydrolysis (saccharification), and fermentation.

A fermentation product may be any substance derived from the fermentation. The fermentation product may be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product may also be protein as a high value product.

In one embodiment, the fermentation product is an alcohol. The term “alcohol” encompasses a substance that contains one or more hydroxyl moieties. The alcohol may be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In another embodiment, the fermentation product is an alkane. The alkane may be an unbranched or a branched alkane. The alkane may be, but is not limited to, pentane, hexane, heptane, octane, nonane, decane, undecane, or dodecane.

In another embodiment, the fermentation product is a cycloalkane. The cycloalkane may be, but is not limited to, cyclopentane, cyclohexane, cycloheptane, or cyclooctane.

In another embodiment, the fermentation product is an alkene. The alkene may be an unbranched or a branched alkene. The alkene may be, but is not limited to, pentene, hexene, heptene, or octene.

In another embodiment, the fermentation product is an amino acid. The organic acid may be, but is not limited to, aspartic acid, glutamic acid, glycine, lysine, serine, or threonine. See, for example, Richard and Margaritis, 2004, Biotechnology and Bioengineering 87(4): 501-515.

In another embodiment, the fermentation product is a gas. The gas may be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another embodiment, the fermentation product is isoprene.

In another embodiment, the fermentation product is a ketone. The term “ketone” encompasses a substance that contains one or more ketone moieties. The ketone may be, but is not limited to, acetone.

In another embodiment, the fermentation product is an organic acid. The organic acid may be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another embodiment, the fermentation product is polyketide.

### Recovery

The fermentation product(s) may be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % may be obtained, which may be used as, for example, fuel ethanol, drinking ethanol, i.e., potable neutral spirits, or industrial ethanol.

The invention is further defined by the following paragraphs:
[1] A process of improving a glucose or xylose yield of saccharification of a lignocellulosic material, the process comprising the steps of:
   a) a first stage comprising saccharifying a lignocellulosic material in a continuous reactor with a first enzyme composition comprising a xylanase, a beta-xylosidase and an endoglucanase; and
   b) a second stage comprising continuing saccharification of the lignocellulosic material, comprising combining the material of step a) with a second enzyme composition comprising one or more cellulases to form a hydrolyzate wherein the hydrolyzate has a glucose yield or a xylose yield that is improved as compared to the yield from a process comprising a single saccharification step.
[2] The process of paragraph 1, wherein the lignocellulosic material has been subjected to a pretreatment method selected from steam explosion and liquid hot water treatment, or a combination thereof.
[3] The process of paragraph 1, wherein the lignocellulosic material has been subjected to a pretreatment selected from chemical pretreatment and mechanical pretreatment, but not subjected to steam explosion or liquid hot water treatment.
[4] The process of paragraph 1, wherein the lignocellulosic material is wheat straw.
[5] The process of paragraph 4, wherein the lignocellulosic material is steam exploded wheat straw.
[6] The process of any of paragraphs 1 to 5, wherein the amount of xylanase in the first enzyme composition is about 4.3 U to about 716.1 U per gram of the lignocellulosic material.
[7] The process of any of paragraphs 1 to 6, wherein the amount of beta-xylosidase in the first enzyme composition is about 0.005 U to about 0.86 U per gram of the lignocellulosic material.
[8] The process of any of paragraphs 1 to 7, wherein the amount of endoglucanase in the first enzyme composition is about 2.84 U to about 117.2 U per gram of the lignocellulosic material.
[9] The process of any of paragraphs 1 to 8, wherein the ratio of enzyme protein of the first enzyme composition to enzyme protein of the second enzyme composition is about 1:2.
[10] The process of any of paragraphs 1 to 9, wherein the first enzyme composition comprises a xylanase selected from the group consisting of: (i) a xylanase comprising or consisting of the mature polypeptide of SEQ ID NO: 12; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 12; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 11; (iv) a xylanase encoded by a polynucleotide that hybridizes under at least high stringency conditions, e.g., very high stringency conditions, with the mature polypeptide coding sequence of SEQ ID NO: 11 or the full-length complement thereof; (v) a xylanase comprising or consisting of the mature polypeptide of SEQ ID NO: 14; (vi) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 14; (vii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 13; (viii) a xylanase encoded by a polynucleotide that hybridizes under at least high stringency conditions, e.g., very high stringency conditions, with the mature polypeptide coding sequence of SEQ ID NO: 13 or the full-length complement thereof; (ix) a xylanase comprising or consisting of the mature polypeptide of SEQ ID NO: 22; (x) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 22; (xi) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 21; and (xii) a xylanase encoded by a polynucleotide that hybridizes under at least high stringency conditions, e.g., very high stringency conditions, with the mature polypeptide coding sequence of SEQ ID NO: 21 or the full-length complement thereof.
[11] The process of any of paragraphs 1 to 10, wherein the first enzyme composition comprises a beta-xylosidase selected from the group consisting of: (i) a beta-xylosidase comprising or consisting of the mature polypeptide of SEQ ID NO: 16; (ii) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 16; (iii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 15; (iv) a beta-xylosidase encoded by a polynucleotide that hybridizes under at least high stringency conditions, e.g., very high stringency conditions, with the mature polypeptide coding sequence of SEQ ID NO: 15 or the full-length complement thereof; (v) a beta-xylosidase comprising or consisting of the mature polypeptide of SEQ ID NO: 24; (vi) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 24; (vii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 23; and (viii) a beta-xylosidase encoded by a polynucleotide that hybridizes under at least high stringency conditions, e.g., very high stringency conditions, with the mature polypeptide coding sequence of SEQ ID NO: 23 or the full-length complement thereof.
[12] The process of any of paragraphs 1 to 11, wherein the first enzyme composition comprises an endoglucanase selected from the group consisting of: (i) an endoglucanase comprising or consisting of the mature polypeptide of SEQ ID NO: 20; (ii) an endoglucanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 20; (iii) an endoglucanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 19; and (iv) an endoglucanase encoded by a polynucleotide that hybridizes under at least high stringency conditions, e.g., very high stringency conditions, with the mature polypeptide coding sequence of SEQ ID NO: 19 or the full-length complement thereof.
[13] The process of any of paragraphs 1 to 12, wherein the second enzyme composition is added at least about 2 hours, at least about 3 hours, at least about 5 hours, at least about 10 hours or at least about 20 hours after combination of the lignocellulosic material and the first enzyme composition.
[14] The process of any of paragraphs 1 to 13, wherein step a) is performed at a pH of about 3.5 to about 5.5.
[15] The process of any of paragraphs 1 to 14, wherein step b) is performed at a pH of about 3.5 to about 5.5.
[16] The process of any of paragraphs 1 to 15, wherein step a) is performed at a lower pH than the pH of step b).
[17] The process of any of paragraphs 1 to 16, wherein step a) is performed in a continuously stirred tank reactor (CSTR).
[18] The process of any of paragraphs 1 to 17, wherein step b) is carried out in the same reactor as step a).
[19] The process of any of paragraphs 1 to 17, wherein step b) is carried out in a separate reactor from step a).
[20] The process of paragraph 19, wherein the separate reactor is in series with the reactor from step a).
[21] The process of paragraph 20, wherein the separate reactor is a batch reactor.
[22] The process of paragraph 20, wherein the separate reactor is a continuously stirred tank reactor (CSTR).
[23] A process of producing a fermentation product from a lignocellulosic material, the process comprising the steps of:

a) hydrolyzing the lignocellulosic material according to the process of any of paragraphs 1 to 22, and

b) fermenting the hydrolyzate to produce a fermentation product.
[24] A process of multi-stage hydrolysis of a lignocellulosic material, the process comprising the steps of:

a) a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U, per gram of the lignocellulosic material; and

b) a second stage comprising continuing saccharification of the lignocellulosic material, comprising combining the material of step a) with a second enzyme composition comprising one or more cellulases.
[25] The process of paragraph 24, wherein the lignocellulosic material has been subjected to a pretreatment method selected from steam explosion and liquid hot water treatment, or a combination thereof.
[26] The process of paragraph 24, wherein the lignocellulosic material has been subjected to a pretreatment selected from chemical pretreatment and mechanical pretreatment, but not subjected to steam explosion or liquid hot water treatment.
[27] The process of paragraph 24, wherein the lignocellulosic material is wheat straw.
[28] The process of paragraph 27, wherein the lignocellulosic material is steam exploded wheat straw.
[29] The process of any of paragraphs 24 to 28, wherein step a) is performed in a continuous reactor.
[30] The process of paragraph 29, wherein step a) is performed in a continuously stirred tank reactor (CSTR).
[31] A process of producing a fermentation product from a lignocellulosic material, the process comprising the steps of:

a) hydrolyzing the lignocellulosic material, comprising:
1) a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U per gram of the lignocellulosic material; and
2) a second stage comprising continuing saccharification of the lignocellulosic material, comprising combining the material of step a) with a second enzyme composition comprising one or more cellulases to form a hydrolyzate; and

b) fermenting the hydrolyzate to produce a fermentation product.
[32] The process of paragraph 31, wherein the pretreated lignocellulosic material has been subjected to a pretreatment method selected from steam explosion and liquid hot water treatment, or a combination thereof.
[33] The process of paragraph 31, wherein the lignocellulosic material has been subjected to a pretreatment selected from chemical pretreatment and mechanical pretreatment, but not subjected to steam explosion or liquid hot water treatment.
[34] The process of paragraph 31, wherein the lignocellulosic material is wheat straw.
[35] The process of paragraph 34, wherein the lignocellulosic material is steam exploded wheat straw.
[36] The process of any of paragraphs 31 to 35, wherein step a) is performed in a continuous reactor.
[37] The process of paragraph 36, wherein step a) is performed in a continuously stirred tank reactor (CSTR).

The present invention is further described by the following examples that should not be construed as limiting the scope of the invention.

The following are referred to in the examples:

Cellulolytic Enzyme Preparation A (“CPrepA”): Cellulolytic enzyme composition derived from Trichoderma reesei further comprising an AA9 (GH61) polypeptide having cellulolytic enhancing activity of SEQ ID NO: 10, a beta-glucosidase of SEQ ID NO: 4, a cellobiohydrolase I of SEQ ID NO: 6, a cellobiohydrolase II of SEQ ID NO: 8, a xylanase of SEQ ID NO: 12, and a beta-xylosidase of SEQ ID NO: 16.

Xylanase Enzyme Preparation A (“XPrepA”): Enzyme composition from Trichoderma reesei, further comprising a xylanase of SEQ ID NO: 12 and a beta-xylosidase of SEQ ID NO: 16.

Xylanase Enzyme Preparation B (“XPrepB”): Enzyme composition from Trichoderma reesei, further comprising a xylanase of SEQ ID NO: 14 herein and a beta-xylosidase of SEQ ID NO: 18.

Endoqlucanase Enzyme Preparation (“EG1”): Enzyme composition from A. oryzae, further comprising an endoglucanase of SEQ ID NO: 20.

The invention described and claimed herein is not to be limited in scope by the specific aspects or embodiments herein disclosed, since such are intended as illustrations of several aspects or embodiments of the invention. Any equivalent aspects or embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### EXAMPLES

### Example 1

### Comparison of Two Stage Hydrolysis with First Step of CSTR vs. Batch

Wheat straw was introduced into a continuous reactor and subjected to a soaking treatment at a temperature of 158° C. for 65 minutes. The soaked mixture was separated in a soaked liquid and a fraction containing the solid soaked raw material by means of a press. The fraction containing the solid soaked raw material was subjected to steam explosion at a temperature of 200° C. for a time of 4 minutes to produce a solid stream.

Soaked liquid was subjected to a concentration step by means of a membrane filtration step, which also removes a portion of acetic acid. First, soaked liquids were subjected to a preliminary pre-separation step to remove solids, by means of centrifugation and macro filtration (bag filter with filter size of 1 micron). Centrifugation was performed by means of an Alfa Laval CLARA 80 centrifuge at 8000 RPM. The soaked liquid was then subjected to concentration by means of an Alfa Laval 2.5″ equipment (membrane code NF99 2517/48), operated at a VCR (Volume Concentration Ratio) of 2.5.

The pre-treatment, including concentration step, produced a soaked liquid and a solid stream in a ratio of liquid stream: solid stream by weight of 1:1. The soaked liquid and the solid stream were used as pre-treated wheat straw material in the following enzymatic hydrolysis experiments.

The dry matter of the soaked liquid after concentration was 10%. pH of the solid stream was 4 and pH of the liquid stream was 4.

The pretreated wheat straw was subjected to one of two hydrolysis reactions: two stage hydrolysis, with the first stage in CSTR and the second stage in batch, as compared to a pure batch hydrolysis.

A first stage CSTR contained a total reaction mass of 100 kg and was operated by discharging 10 kg of hydrolysis reaction material every hour and immediately adding 10 kg of pre-treated wheat straw material and water as 3.2 kg soaked liquid, 4 kg solid stream and 2.8 kg water. At the time of each addition of new material to the CSTR, 35 g of CPrepA was added to the CSTR at a dose of 5.7% (weight/weight glucan). The CSTR retention time was 10 hour. pH was controlled at a targeted 5.2 in the CSTR with additions of 2M sodium hydroxide. The subsequent batch hydrolysis was performed in a Labfors 5 BioEtOH reactor (Infors AG, Switzerland). The reaction in the CSTR was performed at 18% dry matter, 50° C. and pH 5.0 and the reaction in the Labfors 5 BioEtOH reactor was performed at 18% dry matter, 50° C. and pH 5.0. No additional enzymes were added in the subsequent stage batch hydrolysis.

The pure batch hydrolysis was performed in a mixed tank reactor filled with a total of 15 kg reaction mass. CPrepA was added at the start of the reaction in a dose of 5.6% (weight/weight glucan). The reaction was performed at 21% dry matter, 50° C. and pH 5.0.

The hydrolysis performance was evaluated in terms of glucose yield and xylose yield. The total glucose and xylose concentrations from each reaction were determined by HPLC. Glucose yield is the percent ratio of the amount of glucose in the hydrolyzed mixture to the amount of glucans in the pretreated streams, expressed as glucose equivalents. Glucose equivalents were calculated including insoluble glucans, gluco-oligomers, cellobiose and glucose, present in both the solid and liquid of the lignocellulosic biomass, taking into account the different molecular weights. Equivalently, xylose yield is the percent ratio of the amount of xylose in the hydrolyzed mixture to the amount of xylans in the pretreated streams, expressed as xylose equivalents. Xylose equivalents were calculated including insoluble xylans, xylooligomers, xylobiose and xylose, present in both the solid and liquid of the lignocellulosic biomass, taking into account the different molecular weights.

Table 1 shows glucose and xylose yield at different time points of the enzymatic hydrolysis of pre-treated wheat straw using two-stage enzyme addition in a continuously stirred tank reactor followed by hydrolysis in batch (CSTR+batch) compared to hydrolysis in only batch (pure batch) with CPrepA.

A lower glucose yield was obtained with a first stage hydrolysis in a CSTR, followed by second stage hydrolysis in a batch reactor, as compared to a pure batch hydrolysis. Results are set forth in Table 1 and illustrated in FIG. 1. The hydrolysis of the CSTR+batch reaction reached a glucose yield of 42% and a xylose yield of 62% after 82 h of reaction time compared to a glucose yield of 55% and a xylose yield of 65% for the pure batch hydrolysis after 72 h of reaction time. These yields illustrated that resulting sugar yields are lower in first stage CSTR as compared to pure batch.

TABLE 1

Time (h)

| | | 10 h | 34 h | 58 h | 82 h |
|---|---|---|---|---|---|
| CSTR + | Glucose yield | 17% | 34% | 38% | 42% |
| batch | Xylose yield | 30% | 50% | 56% | 62% |

Time (h)

| | | 0 h | 24 h | 48 h | 72 h |
|---|---|---|---|---|---|
| Pure | Glucose yield | 0% | 41% | 50% | 53% |
| batch | Xylose yield | 2% | 52% | 62% | 66% |

Analytical measurements were performed according to the following standards issued by the National Renewable Energy Laboratory (NREL):

Determination of Structural Carbohydrates and Lignin in Biomass; Laboratory Analytical Procedure (LAP) Issue Date: Apr. 25, 2008; Technical Report NREL/TP-510-42618 Revised April 2008

Determination of Extractives in Biomass; Laboratory Analytical Procedure (LAP) Issue Date: Jul. 17, 2005; Technical Report NREL/TP-510-42619 January 2008

Preparation of Samples for Compositional Analysis; Laboratory Analytical Procedure (LAP) Issue Date: Sep. 28, 2005; Technical Report NREL/TP-510-42620 January 2008

Determination of Total Solids in Biomass and Total Dissolved Solids in Liquid Process Samples; Laboratory Analytical Procedure (LAP) Issue Date: Mar. 31, 2008; Technical Report NREL/TP-510-42621 Revised March 2008

Determination of Ash in Biomass; Laboratory Analytical Procedure (LAP) Issue Date: Jul. 17, 2005; Technical Report NREL/TP-510-42622 January 2008

Determination of Sugars, By products, and Degradation Products in Liquid Fraction Process Samples; Laboratory Analytical Procedure (LAP) Issue Date: Dec. 8, 2006; Technical Report NREL/TP-510-42623 January 2008

Determination of Insoluble Solids in Pretreated Biomass Material; Laboratory Analytical Procedure (LAP) Issue Date: Mar. 21, 2008; NREL/TP-510-42627 March 2008

### Example 2

### Viscosity Reduction with Endoglucanase

The cellulosic material used for this experiment was corn stover pretreated by steam explosion in the presence of dilute sulfuric acid. The pretreated cellulosic material was supplied by the National Renewable Energy Laboratory (NREL) in Golden, Colo. Total solid content was 21% and the sulfuric acid concentration was approximately 0.8%. The cellulosic material was heated to 180° C. for approximately 5 minutes before being discharged and steam exploded. The pH of the pretreated substrate was adjusted to 5.0 with sodium hydroxide before enzymes were added. After pH adjustment, the total solids content was 22.5%, due to the added salts.

The reaction was run in a Rapid Visco Analyzer RVA-4 (Newport Scientific Pty. Ltd., ½ Apollo Street, Warriewood, NSW 2102, Australia). This instrument provides a temperature controlled cylindrical compartment with a paddle agitator. Viscosity is deduced from the measured torque of the agitator. The agitation speed was 500 RPM, and the temperature was 50° C.

26.7 g of substrate was added to the aluminum canister used for RVA analysis. 3.3 ml of de-ionized water and enzyme dilution was added to substrate. The resulting total solids content was 20%. The RVA agitator was used to provide slight initial mixing, and then the canister with paddle was added to the RVA for measurement of viscosity during hydrolysis. The experiment was run with 1 and 5 mg enzyme protein per gram cellulose, respectively, for Cellulolytic Enzyme Preparation A (“CPrepA”), and 1 mg enzyme protein per gram cellulose for the Endoglucanase Enzyme Preparation (“EG1”).

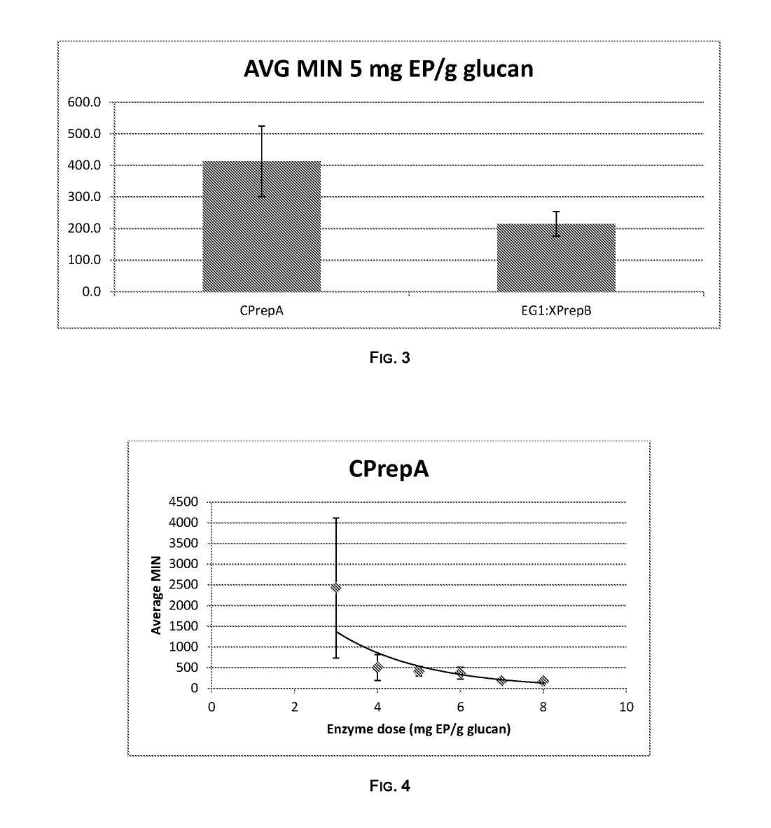

FIG. 2 shows the measured viscosity of the biomass slurry versus time for the three different enzyme dosages. It is seen, that over the course of five hours, a dose of 1 mg enzyme protein per gram cellulose of CPrepA resulted in a final viscosity of approximately 250 cP. A dose of 5 mg enzyme protein per gram cellulose of CPrepA resulted in a final viscosity of approximately 200 cP. A dose of 1 mg enzyme protein per gram cellulose of the EG1 resulted in a final viscosity of approximately 200 cP, and the rate of viscosity reduction was faster than for 5 mg CPrepA. Hence, it is evident that the EG1 is more efficient at reducing the viscosity of a biomass slurry than CPrepA.

### Example 3

### Viscosity Reduction with Enzyme Blend of Endoglucanase and Xylanases

Hydrolysis was performed on pre-treated wheat straw in order to measure apparent viscosity in the biomass slurry. A combination of XPrepB and EG1 was compared to CPrepA with the aim of obtaining more or equal viscosity reduction as a 5 mg EP/g glucan dose of CPrepA.

The fiber fraction and the liquid fraction of pre-treated wheat straw were combined in a ratio of Liquid/Solid=0.75. During mixing of the solid and liquid fraction the pH of the substrate was adjusted to 5.2 with 2 M potassium hydroxide.

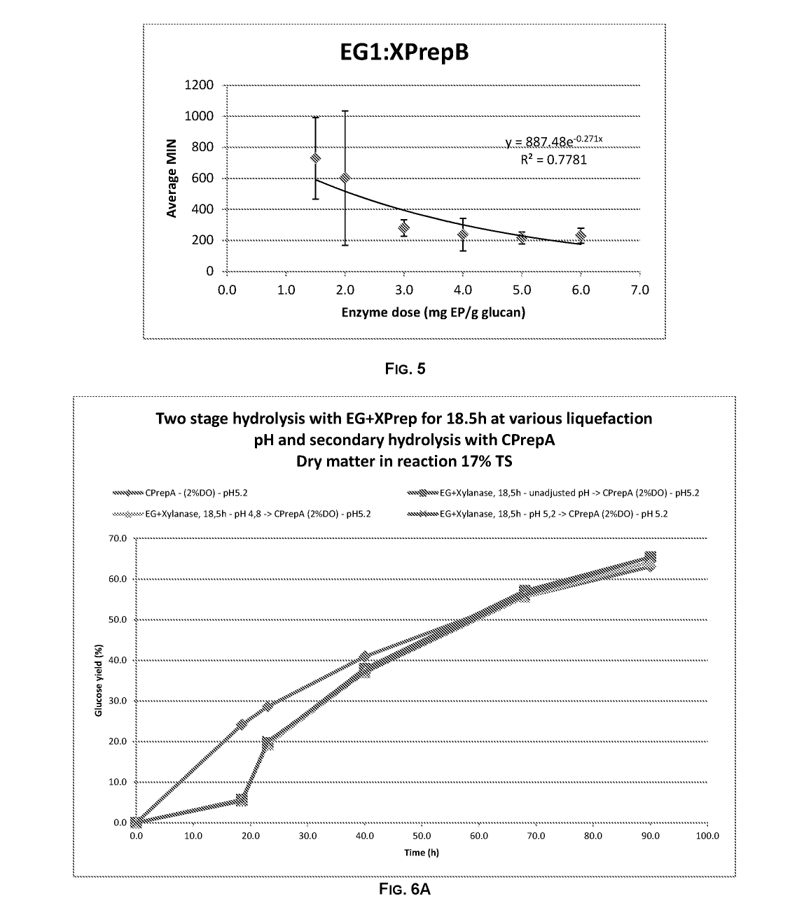

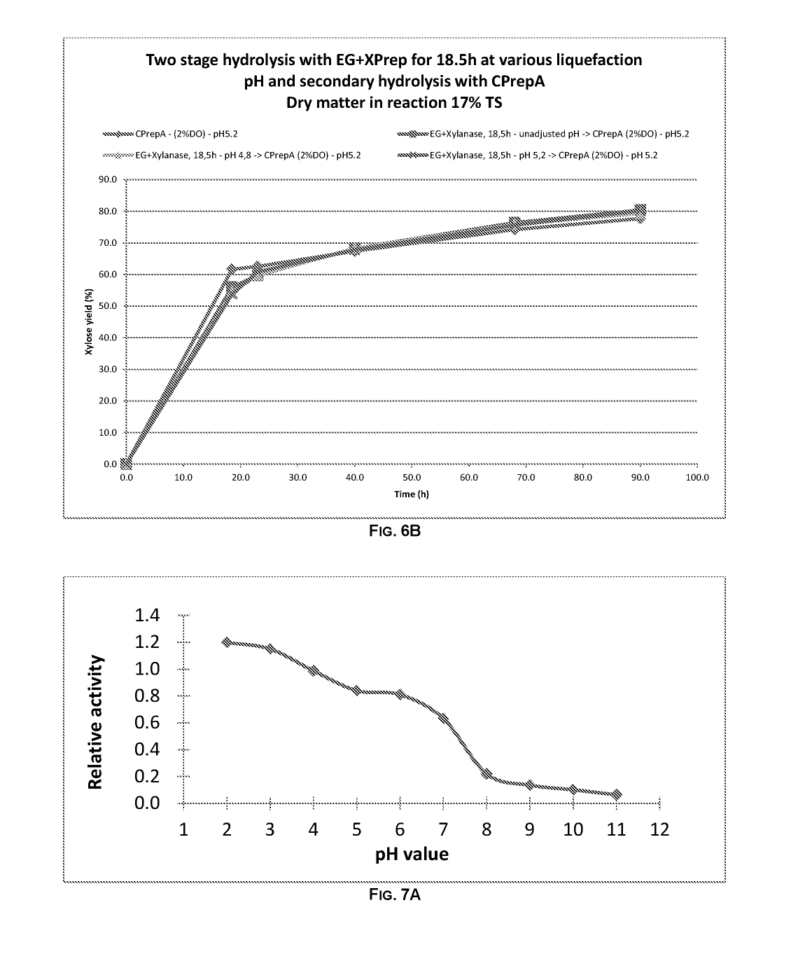

Hydrolysis was started by weighing the mixed and pH adjusted substrate in to 50 ml centrifuge tubes (LOT:525-0160, VWR International, Radnor, Pa., US) that correspond to a final reaction dry matter of 10%. 1 ml of 1M sodium acetate buffer at pH 5.2 was added to each tube. Double deionized water was added to all tubes so that the final weight of all tubes would be 20 g after enzyme addition. To aid mixing in the tubes three stainless steel balls (LOT:412-3141, VWR International, Radnor, Pa., US) was added to all tubes. The reaction in each tube was started by adding CPrepA or the combination of XPrepB and EG1. CPrepA was dosed at 3, 4, 5, 6, 7 and 8 mg EP/g glucan. A 50:50 blend of XPrepB and EG1 was added at 1.5, 2, 3, 4, 5, 6 mg EP/g glucan. Each tube was placed in a FinePCR Thermo Rotisserie Incubators (A. Daigger & Company, Vernon Hills, Ill.) set to 50° C. Tubes were incubated for 6 h and after which the apparent viscosity was measured using a ViPr viscosimeter as described in (WO 2011/107472 A1). For this evaluation the pressure measurement obtained during the aspiration phase of the ViPr viscometer was used (MIN). Evaluation of the result showed that at an enzyme dose of 5 mg EP/g glucan a significantly (P≦0.0072) lower apparent viscosity was obtained using the 50:50 mixture of XPrepB and EG1 when compared to the 5 mg EP/g glucan of CPrepA. Results are shown in FIG. 3, FIG. 4 and FIG. 5.

| Average at 5 mgEP/g | AVG MIN | Stdev MIN | T-Test* |
|---|---|---|---|
| CPrepA | 413 | 112 | 0.0072 |
| EG1:XPrepB | 215 | 39 | |

*T-Test performed as 2 tailed distribution with equal variance of samples

### Example 4

### Two-Stage Dosing of Enzymes in Continuously Stirred Tank Reactors Followed by Batch Reactor

### Pretreatment

Wheat straw was introduced into a continuous reactor and subjected to a soaking treatment at a temperature of 158° C. for 65 minutes. The soaked mixture was separated in a soaked liquid and a fraction containing the solid soaked raw material by means of a press. The fraction containing the solid soaked raw material was subjected to steam explosion at a temperature of 200° C. for a time of 4 minutes to produce a solid stream.

Soaked liquid was subjected to a concentration step by means of a membrane filtration step, which also removes a portion of acetic acid.

First, soaked liquids were subjected to a preliminary pre-separation step to remove solids, by means of centrifugation and macro filtration (bag filter with filter size of 1 micron). Centrifugation was performed by means of an Alfa Laval CLARA 80 centrifuge at 8000 RPM.

The soaked liquid was then subjected to concentration by means of an Alfa Laval 2.5″ equipment (membrane code NF99 2517/48), operated at a VCR (Volume Concentration Ratio) of 2.5.

The pre-treatment, including concentration step, produced a soaked liquid and a solid stream in a ratio liquid stream:solid stream by weight of 1:1.

The soaked liquid and the solid stream were used in the enzymatic hydrolysis experiments. pH of the solid stream was 4 and pH of the liquid stream was 4.

The dry matter of the soaked liquid after concentration was 10%.

### Hydrolysis

An enzymatic hydrolysis in a CSTR+batch configuration was performed by using CPrepA, XPrepB (xylanase: 217±4.8 U/mg; beta-xylosidase: 0.26±0.02 U/mg), and EG1 (cellulase: 71±12 U/mg). The hydrolysis was conducted sequentially in a CSTR, followed by a batch reactor. On a protein dose basis the XPrepB was dosed in a 2:1 ratio over EG1. XPrepB and EG1 comprised one third of the total protein loading in the hydrolysis process and was added into the CSTR at the start of the hydrolysis. CPrepA, used in the second enzyme addition, comprised the remaining two thirds of the total protein.

The CSTR contained a total reaction mass of 200 kg and was operated by discharging 10 kg of hydrolysis reaction material every hour and immediately adding 10 kg of pre-treated wheat straw material and water. At the time of each addition of new material to the CSTR, 24.3 g of EG1 and 6.1 g of XPrepB was added to the CSTR. The CSTR retention time was 20 hour. The subsequent batch hydrolysis was performed in a Labfors 5 BioEtOH reactor (Infors AG, Switzerland). The reaction in the CSTR was performed at 21% dry matter, 50° C. and pH 4.9 and the reaction in the Labfors 5 BioEtOH reactor was performed at 21% dry matter, 50° C. and pH 5.0. The second dose of enzyme, CPrepA (22.2 g), was added after 20 hours of incubation in the Labfors 5 BioEtOH reactor.

As a control, a one stage batch hydrolysis was performed in a mixed tank reactor filled with a total of 15 kg reaction mass. CPrepA was added at the start of the reaction. The reaction was performed at 22% dry matter, 50° C. and pH 5.0.

Equal amounts of enzyme protein per gram of glucan was dosed in both the two stage CSTR followed by batch hydrolysis experiment and the one stage batch hydrolysis experiment.

Regulation of pH was made with additions of 2M sodium hydroxide.

Glucose and xylose concentration in the hydrolysis was determined by HPLC.

The hydrolysis performance was evaluated in terms of glucose yield and xylose yield. Glucose yield is the percent ratio of the amount of glucose in the hydrolyzed mixture to the amount of glucans in the pretreated streams, expressed as glucose equivalents. Glucose equivalents were calculated including insoluble glucans, gluco-oligomers, cellobiose and glucose, present in both the solid and liquid of the lignocellulosic biomass, taking into account the different molecular weights. Equivalently, xylose yield is the percent ratio of the amount of xylose in the hydrolyzed mixture to the amount of xylans in the pretreated streams, expressed as xylose equivalents. Glucose equivalents were calculated including insoluble xylans, xylo-oligomers, xylobiose and xylose, present in both the solid and liquid of the lignocellulosic biomass, taking into account the different molecular weights.

Table 2 shows glucose and xylose yield at different time points of the enzymatic hydrolysis of pre-treated wheat straw using two-stage enzyme addition in a continuously stirred tank reactor followed by hydrolysis in batch (CSTR+batch) compared to hydrolysis in only batch (Pure batch) with CPrepA.

The hydrolysis of the two stage enzyme dosing in CSTR+batch reached a glucose yield of 54% and a xylose yield of 59% after 70 h of reaction time compared to a glucose yield of 50% and a xylose yield of 63% for the one stage enzyme dosing in batch hydrolysis.

TABLE 2

| | | | | | | CSTR + | | |
|---|---|---|---|---|---|---|---|---|
| | | | | | | Batch (EG1 + | | |
| | Pure batch (CPrepA) | | | | | XPrepB + CPrepA) | | |
| | Time (h) | | | | | | | |
| | 0 | 10 | 24 | 48 | 72 | 20 | 40 | 70 |
| glucose yield | 0% | 23% | 36% | 47% | 50% | 11% | 16% | 54% |
| xylose yield | 7% | 50% | 58% | 62% | 63% | 28% | 38% | 59% |

Analytical measurements were performed according to the following standards issued by the National Renewable Energy Laboratory (NREL):

Determination of Structural Carbohydrates and Lignin in Biomass; Laboratory Analytical Procedure (LAP) Issue Date: Apr. 25, 2008; Technical Report NREUTP-510-42618 Revised April 2008

Determination of Extractives in Biomass; Laboratory Analytical Procedure (LAP) Issue Date: Jul. 17, 2005; Technical Report NRELJTP-510-42619 January 2008

Preparation of Samples for Compositional Analysis; Laboratory Analytical Procedure (LAP) Issue Date: Sep. 28, 2005; Technical Report NREL/TP-510-42620 January 2008

Determination of Total Solids in Biomass and Total Dissolved Solids in Liquid Process Samples; Laboratory Analytical Procedure (LAP) Issue Date: Mar. 31, 2008; Technical Report NREL/TP-510-42621 Revised March 2008

Determination of Ash in Biomass; Laboratory Analytical Procedure (LAP) Issue Date: Jul. 17, 2005; Technical Report NRELITP-510-42622 January 2008

Determination of Sugars, By products, and Degradation Products in Liquid Fraction Process Samples; Laboratory Analytical Procedure (LAP) Issue Date: Dec. 8, 2006; Technical Report NREL/TP-510-42623 January 2008

Determination of Insoluble Solids in Pretreated Biomass Material; Laboratory Analytical Procedure (LAP) Issue Date: Mar. 21, 2008; NREUTP-510-42627 March 2008

### Example 5

### Two-Stage Dosing of Enzymes in Infors Reactor

Hydrolysis was performed on pre-treated wheat straw (pretreated as described in Example 4 above) in Labfors 5 BioEtOH reactors (Infors AG, Switzerland). The reactors were filled with a total reaction weight of 1200 g. The fiber fraction and the liquid fraction of pre-treated wheat straw was combined in a ratio of Liquid/Solid=0.75. Substrate was added to a final concentration of 17% dry matter. Temperature, pH, mixing speed and dissolved oxygen concentration (DO) was online controlled by the Labfors reactor. The conditions during hydrolysis were; 50° C., 50 RPM and 2% dissolved oxygen. The pH during the initial 18.5 h was varied at 5.2, 4.8 or unadjusted. The reactors were started up by first mixing the substrate solid and liquid fraction with calculated amount of water. After this the DO was set to 0% by helium sparging into the reactor. After this the reactor was heated to 50° C. and pH was set to desired reaction set point. After this the first dose of enzymes was added. After 18 h liquefaction the DO was set to 2% for all reactors and the second dose of enzymes were added to the reactors. In this experiment four reactors were used and the experimental design was as follows:
R1: single dose: 9 mg EP/g glucan of CPrepA at 2% DO and pH 5.2
R2: first dose: 1 mg EG1+2 mg XPrepB without pH control during the first 18.5 h, second dose: 6 mg EP/g glucan CPrepA at 2% DO, pH 5.2
R3: first dose: 1 mg EG1+2 mg XPrepB and pH 4.8 during the first 18.5 h, second dose: 6 mg EP/g glucan CPrepA at 2% DO, pH 5.2
R4: first dose: 1 mg EG1+2 mg XPrepB and pH 5.2 during the first 18.5 h, second dose: 6 mg EP/g glucan CPrepA at 2% DO and maintained pH 5.2

In the reactor without pH adjustment (R2) the pH was observed to be approximately 4.7 during the initial 18.5 h. In the reactor with only CPrepA (R1) all enzymes were added at the beginning of the reaction.

The total reaction time in this experiment was 90 hours. Samples taken throughout the reactions were diluted by weight and analyzed on HPLC for glucose and xylose concentration (g sugar/kg of slurry). Based on the composition the theoretical amount glucose and xylose release was calculated as g sugar/kg of slurry for the 17% dry matter reaction. The sugar yields were calculated by dividing HPLC measured sugar with the calculated theoretical max sugar.

Quantification of glucose and xylose was performed by high pressure liquid chromatography using a AMINEX® HPX-87H columns (Bio-Rad Laboratories, Hercules, Calif., USA) with an inlet filter (Rheodyne 0.5 μm filter size, 3 mm ID, P/N: 7335-010) and a guard column (Micro-Guard Cation H Refill Cartridges, Bio-Rad Laboratories, Hercules, Calif., USA) with a WATERS® 515 Pump, WATERS® MPSA Millipore, WATERS® 717 Plus Autosampler, WATERS® Column Heater Module and WATERS® 2414 RI detector (Waters Corporation, Milford, Mass., USA). The chromatography was performed at 65° C. with a flow of 0.6 ml/minute of 0.005 M sulfuric acid.

The results shows that in batch hydrolysis a two stage hydrolysis with an initial endoglucanse and xylanase hydrolysis for 18.5 h followed by a dose of cellulosic CPrepA, the final glucose and xylose yield after 90 h is similar to when adding the same amount of enzyme as CPrepA from the beginning of the hydrolysis reaction. The results also show that in the reactions where EG1 and XPrepB are added in the first stage, variance of the pH between 5.2 and 4.7 (R3 and R4) does not affect the final yield. (Table 3 and FIG. 6A and FIG. 6B)

TABLE 3

| | | | R2: EG1 + | | R3: EG1 + | | R4: EG1 + | |
|---|---|---|---|---|---|---|---|---|
| | | | XPrepB, | | XPrepB, | | XPrepB, | |
| | | | 18.5 h - | | 18.5 h - | | 18.5 h - | |
| | | | unadjusted | | pH 4.8 -> | | pH 5.2 -> | |
| | R1: CPrepA - | | pH -> CPrepA | | CPrepA | | CPrepA | |
| | (2% DO) - | | (2% DO) - | | (2% DO) - | | (2% DO) - | |
| | pH 5.2 | | pH 5.2 | | pH 5.2 | | pH 5.2 | |
| Time | Glucose | Xylose | Glucose | Xylose | Glucose | Xylose | Glucose | Xylose |
| (h) | yield | yield | yield | yield | yield | yield | yield | yield |
| 0 | 0.0 | 0.0 | 0.0 | 0.0 | 0.0 | 0.0 | 0.0 | 0.0 |
| 18.5 | 24.1 | 61.6 | 5.7 | 55.9 | 5.4 | 54.3 | 5.4 | 53.8 |
| 23 | 28.6 | 62.5 | 19.6 | 59.7 | 19.2 | 59.8 | 20.0 | 60.8 |
| 40 | 40.9 | 67.4 | 37.9 | 68.2 | 37.0 | 68.2 | 37.1 | 67.8 |
| 68 | 55.6 | 74.2 | 57.1 | 76.3 | 55.6 | 75.8 | 56.1 | 75.5 |
| 90 | 63.0 | 77.7 | 65.4 | 80.3 | 64.2 | 79.3 | 65.4 | 80.1 |

### Example 6

### pH and Temperature Profiles of Endoglucanase Preparation EG1

The substrate in this trial was AZCL β-Glucan (Megazyme International Ireland, Bray Business Park, Bray, Co. Wicklow, Ireland). The substrate suspension was prepared as follows. 0.4% AZCL β-glucan was suspended in buffer with addition of 0.01% Triton X-100 by gentle stirring.

For the pH profile, the following assay procedure was applied. The assay buffer was 100 mM succinic acid, 100 mM (4-(2-hydroxyethyl)-1-piperazineethanesulfonic acid) (HEPES), 100 mM N-cyclohexyl-2-aminoethanesulfonic acid (CHES), 100 mM 4-(cyclohexylamino)-1-butanesulfonic acid (CABS), 1 mM calcium dichloride, 150 mM potassium chloride, and 0.01% Triton X-100. The pH values were adjusted to 2.0, 2.5, 3.0, 3.5, 4.0, 5.0, 6.0, 7.0, 8.0, 9.0, 10.0 and 11.0 with hydrocloric acid or sodium hydroxide. 20 μl EG1 enzyme sample and 200 μl substrate suspension were mixed in a microtiter plate and placed on ice before the reaction. The assay was initiated by transferring the microtiter plate to an Eppendorf Thermomixer (Eppendorf AG, Barkhausenweg 1, 22339 Hamburg, Germany. The plate was incubated for 20 minutes at 700 RPM and 45° C. The incubation was stopped by transferring the plate back to the ice bath. Then the plate was centrifuged in an ice cold centrifuge for a few minutes and 100 μl supernatant was transferred to a microtiter plate. Optical density at 595 nm was read as a measure of enzyme activity. The reaction was done in triplicate, and a blind with buffer instead of enzyme sample was included in the assay.

For the temperature profile, the following procedure was applied. The assay buffer was 200 mM tris(hydroxymethyl)aminomethane) (Tris-HCl) buffer pH 7. 30 μl enzyme sample and 200 μl substrate suspension were mixed in a 1.5 ml Eppendorf tube and placed on ice before the reaction. The assay was initiated by transferring the Eppendorf tube to an Eppendorf Thermomixer. The tubes were incubated for 30 minutes at 1400 RPM and the temperatures 15, 20, 30, 40, 50, 60, 70, and 80° C., respectively. The incubation was stopped by transferring the tube back to the ice bath. Then the tube was centrifuged in an ice cold centrifuge for a few minutes, and 200 μl supernatant was transferred to a microtiter plate. Optical density at 595 nm was read as a measure of enzyme activity. The reaction was done in triplicate, and a blind with buffer instead of enzyme sample was included in the assay.

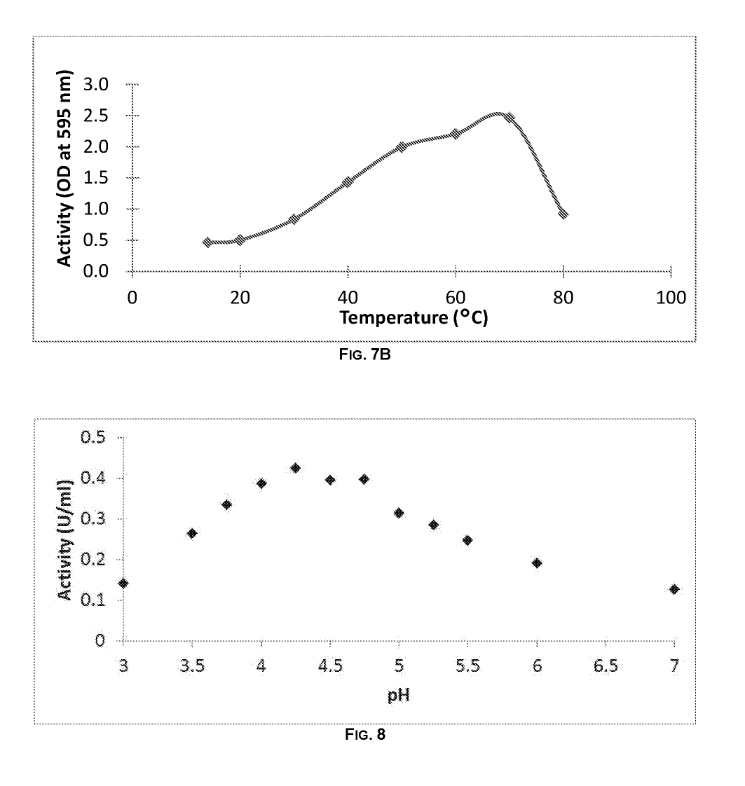

FIG. 7A shows the relative enzyme activity versus pH. The enzyme is active from pH 2 to pH 7, but with highest activity around pH 2-3. It has more than 50% activity at pH 7, as compared to activity at pH 2-3. FIG. 7B shows the relative activity vs. temperature. It can be seen that the enzyme is active at all the tested temperatures (20-80° C.), and with highest activity around 70° C.

### EXAMPLE 7

### pH Profile of Beta-Xylosidase Preparation

An assay for β-xylosidase activity was run with the substrate p-nitrophenyl-β-d-xylopyranoside (Sigma N2132). 2 mM p-nitrophenyl-β-d-xylopyranoside (0.5424 mg/ml) was dissolved in de-ionized water with 0.01% Triton X-100.

An assay buffer was made with 100 mM phosphoric acid, 100 mM acetic acid, 100 mM boric acid, 0.01% Triton X-100, 100 mM potassium chloride, and 2 mM calcium dichloride. pH was adjusted to 3.00, 3.50, 3.75, 4.00, 4.25, 4.50, 4.75, 5.00, 5.25, 5.50, 6.00, and 7.00, respectively, with 27% sodium hydroxide.

A stop solution was made with 0.5 M glycine and 2 mM ethylene diamine tetra-acetic acid (EDTA), adjusted to pH 10.0 with sodium hydroxide.

A dilution of 0.577 mg/l Aspergillus fumigatus β-xylosidase (WO 2011/057140) was made in de-ionized water with 0.01% Triton X-100.

One activity unit, U, is defined by the release of 1 micro mole para-nitrophenol per minute under the conditions of the assay (37° C.). Activity was expressed as U/mg total enzyme. A para-nitrophenol standard curve was run with the concentration range from 0.05-0.50 mM.

20 μl enzyme dilution, 60 μl assay buffer, and 60 μl substrate solution were added to a microtiter plate. The plate was sealed and incubated for 15 minutes at 37° C. in an Eppendorf Thermomixer, shaking at 750 RPM. Then, the plate was refrigerated for 2 minutes, and 100 μl stop solution was added. The optical density at 405 nm was read, and the activity was calculated from the standard curve.

FIG. 8 shows that the optimum pH of this enzyme is found in the range between 4 and 4.75.

### Example 8

### Cellulase Assay

An assay of cellulase was based on the enzymatic endo-hydrolysis of the 1,4-β-D-glucosidic bonds in carboxymethylcellulose (CMC). The products of the reaction β-1,4 glucan oligosaccharides were determined colorimetrically by measuring the resulting increase in reducing groups using a 3,5-dinitrosalicylic acid reagent. Enzyme activity was calculated from the relationship between the concentration of reducing groups, as glucose equivalents, and absorbance at 540 nm.

One unit of cellulase activity is defined as the amount of enzyme which produces 1 micro mole glucose equivalents per minute (U) under the conditions of the assay (pH 5.0 and 50° C.). Activity was expressed as U/mg total enzyme.

### Materials

1.0% (w/v solution) Carboxymethylcellulose (CMC) solution in 50 mM sodium citrate buffer, pH 5.0.

3,5-Dinitrosalicylic acid (DNS) solution: 20 g/L DNS; 20 g/L NaOH; 4 g/L phenol; 1 g/L sodium metabisulphite

Glucose standard solution (1 mg/mL).

### Procedure

The enzyme was diluted with 50 mM sodium citrate buffer (pH 5) ranging from 0.2-0.05 ug of enzyme. A glucose standard curve was made using glucose concentrations of 0.06, 0.12, 0.25, 0.5, and 1 mg/mL. In a PCR plate, 50 μL of enzyme solution was mixed with 50 μL of the CMC substrate and incubated at 50° C. in a thermalcycler. The reaction was stopped after 10 min by addition of 80 μL of DNS solution. This was followed by heating at 95° C. for 10 minutes. Then 130 μL of solution was transferred to a flat bottom clear microplate. The optical density was measured at 540 nm for the different samples and standards.

### Example 9

### Xylanase Assay

An assay of xylanase was based on the enzymatic endo-hydrolysis of the 1,4-β-D-xylosidic bonds in birchwood xylan. The products of the reaction β-1,4 xylan oligosaccharides were determined colorimetrically by measuring the resulting increase in reducing groups using a 3,5-dinitrosalicylic acid reagent. Enzyme activity was calculated from the relationship between the concentration of reducing groups, as xylose equivalents, and absorbance at 540 nm.

One unit of xylanase activity is defined as the amount of enzyme which produces 1 micro mole xylose equivalents per minute (U) under the conditions of the assay (pH 5.0 and 50° C.). Activity was expressed as U/mg total enzyme.

### Materials

1.0% (w/v solution) birchwood xylan solution in 50 mM sodium citrate buffer, pH 5.0.

3,5-Dinitrosalicylic acid (DNS) solution: 20 g/L DNS; 20 g/L NaOH; 4 g/L phenol; 1 g/L sodium metabisulphite

Xylose standard solution (1 mg/mL).

### Procedure

The enzyme was diluted with 50 mM sodium citrate buffer (pH 5) ranging from 0.2-0.05 ug of enzyme. A xylose standard curve was made using xylose concentrations of 0.06, 0.12, 0.25, 0.5, and 1 mg/mL. In a PCR plate, 50 μL of enzyme solution was mixed with 50 μL of the birchwood xylan substrate and incubated at 50° C. in a thermalcycler. The reaction was stopped after 10 min by addition of 80 μL of DNS solution. This was followed by heating at 95° C. for 10 minutes. Then 130 μL of solution was transferred to a flat bottom clear microplate. The optical density was measured at 540 nm for the different samples and standards.

### Example 10

### Beta-Xylosidase Assay

An assay of β-xylosidase was based on the enzymatic hydrolysis of the paranitrophenol-β-D-xylopyranoside (pNPX) substrate. The product of the reaction (paranitrophenol) was determined colorimetrically by measuring the resulting increase in absorption at 410 nm under alkaline conditions. Enzyme activity was calculated from the relationship between product and absorbance at 410 nm.

One unit of β-xylosidase activity is defined as the amount of enzyme which produces 1 micro mole pNP product per minute (U) under the conditions of the assay (pH 5.0 and 50° C.). Activity was expressed as U/mg total enzyme.

### Materials

1 mM pNPX solution in 50 mM sodium citrate buffer, pH 5.0.

pNP standard solution (5 mM).

### Procedure

The enzyme was diluted with 50 mM sodium citrate buffer (pH 5) ranging from 4-0.5 ug enzyme. A pNP standard curve was made using pNP concentrations of 0, 0.03, 0.06, 0.12, 0.25, 0.5, mM. In a microplate, 75 μL of enzyme solution was mixed with 75 μL of the pNPX substrate and incubated at 50° C. The reaction was stopped after 30 min by addition of 100 μL of 0.2 M sodium carbonate solution (pH 11). The optical density was measured at 410 nm for the different samples and standards.

Although the invention has been described with reference to the above examples, it will be understood that modifications and variations are encompassed within the spirit and scope of the invention. Accordingly, the invention is limited only by the following claims.

1. A process of improving a glucose or xylose yield of saccharification of a lignocellulosic material, the process comprising the steps of:
a) a first stage comprising saccharifying a lignocellulosic material in a continuous reactor with a first enzyme composition comprising a xylanase, a beta-xylosidase and an endoglucanase; and
b) a second stage comprising continuing saccharification of the lignocellulosic material, comprising combining the material of step a) with a second enzyme composition comprising one or more cellulases to form a hydrolyzate wherein the hydrolyzate has a glucose yield or a xylose yield that is improved as compared to the yield from a process comprising a single saccharification step.

2. The process of claim 1, wherein the amount of xylanase in the first enzyme composition is about 4.3 U to about 716.1 U per gram of the lignocellulosic material.

3. The process of claim 1, wherein the amount of beta-xylosidase in the first enzyme composition is about 0.005 U to about 0.86 U per gram of the lignocellulosic material.

4. The process of claim 1, wherein the amount of endoglucanase in the first enzyme composition is about 2.84 U to about 117.2 U per gram of the lignocellulosic material.

5. The process of claim 1, wherein the ratio of enzyme protein of the first enzyme composition to enzyme protein of the second enzyme composition is about 1:2.

6. The process of claim 1, wherein the first enzyme composition comprises a xylanase selected from the group consisting of: (i) a xylanase comprising or consisting of the mature polypeptide of SEQ ID NO: 12; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 12; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 11; (iv) a xylanase encoded by a polynucleotide that hybridizes under at least high stringency conditions, e.g., very high stringency conditions, with the mature polypeptide coding sequence of SEQ ID NO: 11 or the full-length complement thereof; (v) a xylanase comprising or consisting of the mature polypeptide of SEQ ID NO: 14; (vi) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 14; (vii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 13; (viii) a xylanase encoded by a polynucleotide that hybridizes under at least high stringency conditions, e.g., very high stringency conditions, with the mature polypeptide coding sequence of SEQ ID NO: 13 or the full-length complement thereof; (ix) a xylanase comprising or consisting of the mature polypeptide of SEQ ID NO: 22; (x) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 22; (xi) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 21; and (xii) a xylanase encoded by a polynucleotide that hybridizes under at least high stringency conditions, e.g., very high stringency conditions, with the mature polypeptide coding sequence of SEQ ID NO: 21 or the full-length complement thereof.

7. The process of claim 1, wherein the first enzyme composition comprises a beta-xylosidase selected from the group consisting of: (i) a beta-xylosidase comprising or consisting of the mature polypeptide of SEQ ID NO: 16; (ii) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 16; (iii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 15; (iv) a beta-xylosidase encoded by a polynucleotide that hybridizes under at least high stringency conditions, e.g., very high stringency conditions, with the mature polypeptide coding sequence of SEQ ID NO: 15 or the full-length complement thereof; (v) a beta-xylosidase comprising or consisting of the mature polypeptide of SEQ ID NO: 24; (vi) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 24; (vii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 23; and (viii) a beta-xylosidase encoded by a polynucleotide that hybridizes under at least high stringency conditions, e.g., very high stringency conditions, with the mature polypeptide coding sequence of SEQ ID NO: 23 or the full-length complement thereof.

8. The process of claim 1, wherein the first enzyme composition comprises an endoglucanase selected from the group consisting of: (i) an endoglucanase comprising or consisting of the mature polypeptide of SEQ ID NO: 20; (ii) an endoglucanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide of SEQ ID NO: 20; (iii) an endoglucanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99% sequence identity to the mature polypeptide coding sequence of SEQ ID NO: 19; and (iv) an endoglucanase encoded by a polynucleotide that hybridizes under at least high stringency conditions, e.g., very high stringency conditions, with the mature polypeptide coding sequence of SEQ ID NO: 19 or the full-length complement thereof.

9. The process of claim 1, wherein step a) is performed in a continuously stirred tank reactor (CSTR).

10. The process of claim 1, wherein step b) is carried out in the same reactor as step a).

11. The process of claim 1, wherein step b) is carried out in a separate reactor from step a).

12. The process of claim 11, wherein the separate reactor is in series with the reactor from step a).

13. The process of claim 12, wherein the separate reactor is a batch reactor.

14. The process of claim 12, wherein the separate reactor is a continuously stirred tank reactor (CSTR).

15. A process of producing a fermentation product from a lignocellulosic material, the process comprising the steps of:
a) hydrolyzing the lignocellulosic material according to the process of claim 1, and
b) fermenting the hydrolyzate to produce a fermentation product.

16. A process of multi-stage hydrolysis of a lignocellulosic material, the process comprising the steps of:
a) a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U, per gram of the lignocellulosic material; and
b) a second stage comprising continuing saccharification of the lignocellulosic material, comprising combining the material of step a) with a second enzyme composition comprising one or more cellulases.

17. A process of producing a fermentation product from a lignocellulosic material, the process comprising the steps of:
a) hydrolyzing the lignocellulosic material, comprising:
1) a first stage comprising saccharifying a lignocellulosic material with a first enzyme composition comprising a xylanase in an amount of about 4.3 U to about 716.1 U per gram of the lignocellulosic material, a beta-xylosidase in an amount of about 0.005 U to about 0.86 U per gram of the lignocellulosic material and an endoglucanase in an amount of about 2.84 U to about 117.2 U per gram of the lignocellulosic material; and
2) a second stage comprising continuing saccharification of the lignocellulosic material, comprising combining the material of step a) with a second enzyme composition comprising one or more cellulases to form a hydrolyzate; and
b) fermenting the hydrolyzate to produce a fermentation product.

18. The process of claim 16, wherein step a) is performed in a continuous reactor.

19. The process of claim 18, wherein step a) is performed in a continuously stirred tank reactor (CSTR).

20. The process of claim 17, wherein step a) is performed in a continuous reactor.


SEQUENCE LISTING (24 sequences)

SEQ ID NO: 1 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 2 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 3 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtgactgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggggcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccgagttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggttacatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 4 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRDSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWGAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTAEFPYLVTPEQAIQREVISNGGNVFAVTDNGALSQMADVASQSSVSLVFVNADSGEGYISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 5 - DNA, 1599 bp, Aspergillus fumigatus
atgctggcctccaccttctcctaccgcatgtacaagaccgcgctcatcctggccgcccttctgggctctggccaggctcagcaggtcggtacttcccaggcggaagtgcatccgtccatgacctggcagagctgcacggctggcggcagctgcaccaccaacaacggcaaggtggtcatcgacgcgaactggcgttgggtgcacaaagtcggcgactacaccaactgctacaccggcaacacctgggacacgactatctgccctgacgatgcgacctgcgcatccaactgcgcccttgagggtgccaactacgaatccacctatggtgtgaccgccagcggcaattccctccgcctcaacttcgtcaccaccagccagcagaagaacattggctcgcgtctgtacatgatgaaggacgactcgacctacgagatgtttaagctgctgaaccaggagttcaccttcgatgtcgatgtctccaacctcccctgcggtctcaacggtgctctgtactttgtcgccatggacgccgacggtggcatgtccaagtacccaaccaacaaggccggtgccaagtacggtactggatactgtgactcgcagtgccctcgcgacctcaagttcatcaacggtcaggccaacgtcgaagggtggcagccctcctccaacgatgccaatgcgggtaccggcaaccacgggtcctgctgcgcggagatggatatctgggaggccaacagcatctccacggccttcaccccccatccgtgcgacacgcccggccaggtgatgtgcaccggtgatgcctgcggtggcacctacagctccgaccgctacggcggcacctgcgaccccgacggatgtgatttcaactccttccgccagggcaacaagaccttctacggccctggcatgaccgtcgacaccaagagcaagtttaccgtcgtcacccagttcatcaccgacgacggcacctccagcggcaccctcaaggagatcaagcgcttctacgtgcagaacggcaaggtgatccccaactcggagtcgacctggaccggcgtcagcggcaactccatcaccaccgagtactgcaccgcccagaagagcctgttccaggaccagaacgtcttcgaaaagcacggcggcctcgagggcatgggtgctgccctcgcccagggtatggttctcgtcatgtccctgtgggatgatcactcggccaacatgctctggctcgacagcaactacccgaccactgcctcttccaccactcccggcgtcgcccgtggtacctgcgacatctcctccggcgtccctgcggatgtcgaggcgaaccaccccgacgcctacgtcgtctactccaacatcaaggtcggccccatcggctcgaccttcaacagcggtggctcgaaccccggtggcggaaccaccacgacaactaccacccagcctactaccaccacgaccacggctggaaaccctggcggcaccggagtcgcacagcactatggccagtgtggtggaatcggatggaccggacccacaacctgtgccagcccttatacctgccagaagctgaatgattattactctcagtgcctgtag

SEQ ID NO: 6 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 7 - DNA, 1713 bp, Aspergillus fumigatus
atgaagcaccttgcatcttccatcgcattgactctactgttgcctgccgtgcaggcccagcagaccgtatggggccaatgtatgttctggctgtcactggaataagactgtatcaactgctgatatgcttctaggtggcggccaaggctggtctggcccgacgagctgtgttgccggcgcagcctgtagcacactgaatccctgtatgttagatatcgtcctgagtggagacttatactgacttccttagactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctgtaagtggccttatcccaatactgagaccaactctctgacagtcgtagcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcggtaggccgtacacctccgttgcgcgccgcctttctctgacatcttgcagaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggtatgcctcacttcccgcattctgtatcccttccagacactaactcatcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctgtaagtgcttattccaatcgccgatgtgtgccgactaatcaatgtttcagcccggaatggcgtccagcccacgaagcaaaacgcctggggtgactggtgcaacgtcatcggcaccggcttcggtgttcgcccctcgactaacaccggcgatccgctccaggatgcctttgtgtggatcaagcccggtggagagagtgatggcacgtccaactcgacttccccccggtatgacgcgcactgcggatatagtgatgctctgcagcctgctcctgaggctggtacttggttccaggtatgtcatccattagccagatgagggataagtgactgacggacctaggcctactttgagcagcttctgaccaacgctaacccgtccttttaa

SEQ ID NO: 8 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 9 - DNA, 835 bp, Penicillium emersonii
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 10 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 11 - DNA, 1415 bp, Aspergillus fumigatus
atggtccatctatcttcattggcagcagccctggctgctctgcctctgtatgtttacccactcacgagaggaggaacagctttgacattgctatagtgtatatggagctggcctgaacacagcagccaaagccaaaggactaaagtactttggttccgccacggacaatccagagctcacggactctgcgtatgtcgcgcaactgagcaacaccgatgattttggtcaaatcacacccggaaactccatgaaggtttgcttacgtctgcctccctggagcattgcctcaaaagctaattggttgttttgtttggatagtgggatgccaccgagccttctcagaattctttttcgttcgcaaatggagacgccgtggtcaatctggcgaacaagaatggccagctgatgcgatgccatactctggtctggcacagtcagctaccgaactggggtatgtaaacgtcttgtctattctcaaatactctctaacagttgacagtctctagcgggtcatggaccaatgcgacccttttggcggccatgaagaatcatatcaccaatgtggttactcactacaaggggaagtgctacgcctgggatgttgtcaatgaaggtttgttgctccatctatcctcaatagttcttttgaaactgacaagcctgtcaatctagccctgaacgaggacggtactttccgtaactctgtcttctaccagatcatcggcccagcatacattcctattgcgttcgccacggctgctgccgcagatcccgacgtgaaactctactacaacgactacaacattgaatactcaggcgccaaagcgactgctgcgcagaatatcgtcaagatgatcaaggcctacggcgcgaagatcgacggcgtcggcctccaggcacactttatcgtcggcagcactccgagtcaatcggatctgacgaccgtcttgaagggctacactgctctcggcgttgaggtggcctataccgaacttgacatccgcatgcagctgccctcgaccgccgcaaagctggcccagcagtccactgacttccaaggcgtggccgcagcatgcgttagcaccactggctgcgtgggtgtcactatctgggactggaccgacaagtactcctgggtccccagcgtgttccaaggctacggcgccccattgccttgggatgagaactatgtgaagaagccagcgtacgatggcctgatggcgggtcttggagcaagcggctccggcaccacaacgaccactactactacttctactacgacaggaggtacggaccctactggagtcgctcagaaatggggacagtgtggcggtattggctggaccgggccaacaacttgtgtcagtggtaccacttgccaaaagctgaatgactggtactcacagtgcctgtaa

SEQ ID NO: 12 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 13 - DNA, 1197 bp, Trichophaea saccata
atgcgtaccttctcgtctcttctcggtgttgcccttctcttgggtgcagctaatgcccaggtcgcggtttggggacagtgtggtggcattggttactctggctcgacaacctgcgctgcgggaacgacttgtgttaagctgaacgactactactcccaatgccaacccggcggtaccactttgacaaccaccaccaaacccgccaccactaccactaccaccacggcaacttctccctcatcttctcccggattaaatgccctggcacaaaagagcggccggtacttcggtagtgcaactgacaacccagagctctccgatgcggcatacattgccatcctgagcaacaaaaacgagtttgggatcatcacgcctggaaactcgatgaaatgggatgctactgaaccgtcccgcgggagtttctcgttcactggtggacagcaaattgttgattttgcgcagggcaatgggcaggctatcagaggccatactcttgtctggtactcccagttgccgtcctgggttactagcggaaacttcgataaagctacattgacatcgatcatgcaaaatcacattacaactcttgtcagccactggaagggccagctcgcctactgggatgttgtcaacgaagcattcaacgatgatggcactttccgtcaaaacgtgttctacacaaccattggagaggactacatccagctcgccttcgaagccgcccgtgccgccgacccgaccgcaaagctctgcatcaacgactacaacatcgagggcactggagccaagtcaacagccatgtacaatctcgtctcgaagctgaaatccgccggcgttcccatcgactgtattggtgttcagggacacctcatcgtcggtgaagttcccaccaccatccaagcaaaccttgcccagtttgcgtctttgggtgtggatgtcgcgatcacggagctagatatcagaatgacgctgccatctacgactgcattgctccagcagcaggctaaggattacgtctcggttgttacagcctgcatgaatgttcccaggtgtatcggtatcaccatctgggactacactgataaatactcttgggtgccacaaaccttcagcggccagggcgatgcttgcccatgggatgccaacctgcagaagaagccagcctactccgctattgcgtctgctcttgcggcttga

SEQ ID NO: 14 - protein, 398 aa, Trichophaea saccata
MRTFSSLLGVALLLGAANAQVAVWGQCGGIGYSGSTTCAAGTTCVKLNDYYSQCQPGGTTLTTTTKPATTTTTTTATSPSSSPGLNALAQKSGRYFGSATDNPELSDAAYIAILSNKNEFGIITPGNSMKWDATEPSRGSFSFTGGQQIVDFAQGNGQAIRGHTLVWYSQLPSWVTSGNFDKATLTSIMQNHITTLVSHWKGQLAYWDVVNEAFNDDGTFRQNVFYTTIGEDYIQLAFEAARAADPTAKLCINDYNIEGTGAKSTAMYNLVSKLKSAGVPIDCIGVQGHLIVGEVPTTIQANLAQFASLGVDVAITELDIRMTLPSTTALLQQQAKDYVSVVTACMNVPRCIGITIWDYTDKYSWVPQTFSGQGDACPWDANLQKKPAYSAIASALAA

SEQ ID NO: 15 - DNA, 2376 bp, Aspergillus fumigatus
atggcggttgccaaatctattgctgccgtgctggtagcactgttgcctggtgcgcttgctcaggcgaatacaagctatgttgattacaatgtggaggcgaatccggatctcacccctcagtcggtcgctacgattgacctgtcctttcccgactgcgagaatggaccgctcagcaagactctcgtttgcgacacgtcggctcggccgcatgaccgagctgctgccctggtttccatgttcaccttcgaggagctggtgaacaacacaggcaacactagccctggtgttccaagacttggtctccctccgtaccaagtatggagcgaggctctccatggacttgaccgcgccaacttcacaaacgagggagagtacagctgggccacctcgttccccatgcctatcctgacaatgtcggccttgaaccgaaccctgatcaaccagatcgcgaccatcatcgcaactcaaggacgagctttcaataacgttgggcggtatgggctggacgtgtacgccccgaatataaatgcattcagatcggctatgtggggaagaggtcaagagacccccggagaagacgcttactgcctggcatcggcgtatgcgtacgagtatatcactggcatccagggtggtgttgatccggaacacctcaagttggtggccactgccaaacactatgcgggctacgatcttgagaactgggacggtcactcccgtttgggcaacgatatgaacattacacagcaggaactttccgaatactacacccctcagttccttgttgcagccagagacgccaaagtgcacagtgtcatgtgctcctacaacgcggtaaatggggtgcccagctgcgcaaactcgttcttcctccagaccctcctccgtgacacattcggcttcgtcgaggatggttatgtatccagcgactgcgactcggcgtacaatgtctggaacccgcacgagtttgcggccaacatcacgggggccgctgcagactctatccgggcggggacggacattgattgcggcactacttatcaatactatttcggcgaagcctttgacgagcaagaggtcacccgtgcagaaatcgaaagaggtgtgatccgcctgtacagcaacttggtgcgtctcggctatttcgatggcaatggaagcgtgtatcgggacctgacgtggaatgatgtcgtgaccacggatgcctggaatatctcatacgaagccgctgtagaaggcattgtcctactgaagaacgatggaaccttgcctctcgccaagtcggtccgcagtgttgcattgattgggccctggatgaatgtgacgactcagcttcagggcaactactttggaccggcgccttatctgattagtccgttgaatgccttccagaattctgacttcgacgtgaactacgctttcggcacgaacatttcatcccactccacagatgggttttccgaggcgttgtctgctgcgaagaaatccgacgtcatcatattcgcgggcgggattgacaacactttggaagcagaagccatggatcgcatgaatatcacatggcccggcaatcagctacagctcatcgaccagttgagccaactcggcaaaccgctgatcgtcctccagatgggcggcggccaagtcgactcctcctcgctcaagtccaacaagaatgtcaactccctgatctggggtggataccccggacaatccggcgggcaggctctcctagacatcatcaccggcaagcgcgcccccgccggccgactcgtggtcacgcagtacccggccgaatacgcaacccagttccccgccaccgacatgagcctgcggcctcacggcaataatcccggccagacctacatgtggtacaccggcacccccgtctacgagtttggccacgggctcttctacacgaccttccacgcctccctccctggcaccggcaaggacaagacctccttcaacatccaagacctcctcacgcagccgcatccgggcttcgcaaacgtcgagcaaatgcctttgctcaacttcaccgtgacgatcaccaataccggcaaggtcgcttccgactacactgctatgctcttcgcgaacaccaccgcgggacctgctccatacccgaacaagtggctcgtcggcttcgaccggctggcgagcctggaaccgcacaggtcgcagactatgaccatccccgtgactatcgacagcgtggctcgtacggatgaggccggcaatcgggttctctacccgggaaagtacgagttggccctgaacaatgagcggtcggttgtccttcagtttgtgctgacaggccgagaggctgtgattttcaagtggcctgtagagcagcagcagatttcgtctgcg

SEQ ID NO: 16 - protein, 792 aa, Aspergillus fumigatus
MAVAKSIAAVLVALLPGALAQANTSYVDYNVEANPDLTPQSVATIDLSFPDCENGPLSKTLVCDTSARPHDRAAALVSMFTFEELVNNTGNTSPGVPRLGLPPYQVWSEALHGLDRANFTNEGEYSWATSFPMPILTMSALNRTLINQIATIIATQGRAFNNVGRYGLDVYAPNINAFRSAMWGRGQETPGEDAYCLASAYAYEYITGIQGGVDPEHLKLVATAKHYAGYDLENWDGHSRLGNDMNITQQELSEYYTPQFLVAARDAKVHSVMCSYNAVNGVPSCANSFFLQTLLRDTFGFVEDGYVSSDCDSAYNVWNPHEFAANITGAAADSIRAGTDIDCGTTYQYYFGEAFDEQEVTRAEIERGVIRLYSNLVRLGYFDGNGSVYRDLTWNDVVTTDAWNISYEAAVEGIVLLKNDGTLPLAKSVRSVALIGPWMNVTTQLQGNYFGPAPYLISPLNAFQNSDFDVNYAFGTNISSHSTDGFSEALSAAKKSDVIIFAGGIDNTLEAEAMDRMNITWPGNQLQLIDQLSQLGKPLIVLQMGGGQVDSSSLKSNKNVNSLIWGGYPGQSGGQALLDIITGKRAPAGRLVVTQYPAEYATQFPATDMSLRPHGNNPGQTYMWYTGTPVYEFGHGLFYTTFHASLPGTGKDKTSFNIQDLLTQPHPGFANVEQMPLLNFTVTITNTGKVASDYTAMLFANTTAGPAPYPNKWLVGFDRLASLEPHRSQTMTIPVTIDSVARTDEAGNRVLYPGKYELALNNERSVVLQFVLTGREAVIFKWPVEQQQISSA

SEQ ID NO: 17 - DNA, 2391 bp, Talaromyces emersonii
atgatgactcccacggcgattctcaccgcagtggcggcgctcctgcccaccgcgacatgggcacaggataaccaaacctatgccaattactcgtcgcagtctcagccggacctgtttccccggaccgtcgcgaccatcgacctgtccttccccgactgtgagaatggcccgctcagcacgaacctggtgtgcaacaaatcggccgatccctgggcccgagctgaggccctcatctcgctctttaccctcgaagagctgattaacaacacccagaacaccgctcctggcgtgccccgtttgggtctgccccagtatcaggtgtggaatgaagctctgcacggactggaccgcgccaatttctcccattcgggcgaatacagctgggccacgtccttccccatgcccatcctgtcgatggcgtccttcaaccggaccctcatcaaccagattgcctccatcattgcaacgcaagcccgtgccttcaacaacgccggccgttacggccttgacagctatgcgcccaacatcaatggcttccgcagtcccctctggggccgtggacaggagacgcctggtgaggatgcgttcttcttgagttccacctatgcgtacgagtacatcacaggcctgcagggcggtgtcgacccagagcatgtcaagatcgtcgcgacggcgaagcacttcgccggctatgatctggagaactggggcaacgtctctcggctggggttcaatgctatcatcacgcagcaggatctctccgagtactacacccctcagttcctggcgtctgctcgatacgccaagacgcgcagcatcatgtgctcctacaatgcagtgaatggagtcccaagctgtgccaactccttcttcctccagacgcttctccgagaaaactttgacttcgttgacgacgggtacgtctcgtcggattgcgacgccgtctacaacgtcttcaacccacacggttacgcccttaaccagtcgggagccgctgcggactcgctcctagcaggtaccgatatcgactgtggtcagaccttgccgtggcacctgaatgagtccttcgtagaaggatacgtctcccgcggtgatatcgagaaatccctcacccgtctctactcaaacctggtgcgtctcggctactttgacggcaacaacagcgagtaccgcaacctcaactggaacgacgtcgtgactacggacgcctggaacatctcgtacgaggccgcggtggaaggtatcaccctgctcaagaacgacggaacgctgccgctgtccaagaaggtccgcagcattgcgctcatcggtccttgggccaatgccacggtgcagatgcagggtaactactatggaacgccaccgtatctgatcagtccgctggaagccgccaaggccagtgggttcacggtcaactatgcattcggtaccaacatctcgaccgattctacccagtggttcgcggaagccatcgcggcggcgaagaagtcggacgtgatcatctacgccggtggtattgacaacacgatcgaggcagagggacaggaccgcacggatctcaagtggccggggaaccagctggatctgatcgagcagctcagccaggtgggcaagcccttggtcgtcctgcagatgggcggtggccaggtggattcgtcgtcactcaaggccaacaagaatgtcaacgctctggtgtggggtggctatcccggacagtcgggtggtgcggccctgtttgacatccttacgggcaagcgtgcgccggccggtcgtctggtgagcacgcagtacccggccgagtatgcgacgcagttcccggccaacgacatgaacctgcgtccgaacggcagcaacccgggacagacatacatctggtacacgggcacgcccgtgtatgagttcggccacggtctgttctacacggagttccaggagtcggctgcggcgggcacgaacaagacgtcgactttcgacattctggaccttttctccacccctcatccgggatacgagtacatcgagcaggttccgttcatcaacgtgactgtggacgtgaagaacgtcggccacacgccatcgccgtacacgggtctgttgttcgcgaacacgacagccgggcccaagccgtacccgaacaaatggctcgtcgggttcgactggctgccgacgatccagccgggcgagactgccaagttgacgatcccggtgccgttgggcgcgattgcgtgggcggacgagaacggcaacaaggtggtcttcccgggcaactacgaattggcactgaacaatgagcgatcggtagtggtgtcgttcacgctgacgggcgatgcggcgactctagagaaatggcctttgtgggagcaggcggttccgggggtgctgcagcaataa

SEQ ID NO: 18 - protein, 796 aa, Talaromyces emersonii
MMTPTAILTAVAALLPTATWAQDNQTYANYSSQSQPDLFPRTVATIDLSFPDCENGPLSTNLVCNKSADPWARAEALISLFTLEELINNTQNTAPGVPRLGLPQYQVWNEALHGLDRANFSHSGEYSWATSFPMPILSMASFNRTLINQIASIIATQARAFNNAGRYGLDSYAPNINGFRSPLWGRGQETPGEDAFFLSSTYAYEYITGLQGGVDPEHVKIVATAKHFAGYDLENWGNVSRLGFNAIITQQDLSEYYTPQFLASARYAKTRSIMCSYNAVNGVPSCANSFFLQTLLRENFDFVDDGYVSSDCDAVYNVFNPHGYALNQSGAAADSLLAGTDIDCGQTLPWHLNESFVEGYVSRGDIEKSLTRLYSNLVRLGYFDGNNSEYRNLNWNDVVTTDAWNISYEAAVEGITLLKNDGTLPLSKKVRSIALIGPWANATVQMQGNYYGTPPYLISPLEAAKASGFTVNYAFGTNISTDSTQWFAEAIAAAKKSDVIIYAGGIDNTIEAEGQDRTDLKWPGNQLDLIEQLSQVGKPLVVLQMGGGQVDSSSLKANKNVNALVWGGYPGQSGGAALFDILTGKRAPAGRLVSTQYPAEYATQFPANDMNLRPNGSNPGQTYIWYTGTPVYEFGHGLFYTEFQESAAAGTNKTSTFDILDLFSTPHPGYEYIEQVPFINVTVDVKNVGHTPSPYTGLLFANTTAGPKPYPNKWLVGFDWLPTIQPGETAKLTIPVPLGAIAWADENGNKVVFPGNYELALNNERSVVVSFTLTGDAATLEKWPLWEQAVPGVLQQ

SEQ ID NO: 19 - DNA, 1008 bp, Thermoascus aurantiacus
atgaagctcggctctctcgtgctcgctctcagcgcagctaggcttacactgtcggcccctctcgcagacagaaagcaggagaccaagcgtgcgaaagtattccaatggttcggttcgaacgagtccggtgctgaattcggaagccagaaccttccaggagtcgagggaaaggattatatatggcctgatcccaacaccattgacacattgatcagcaaggggatgaacatctttcgtgtcccctttatgatggagagattggttcccaactcaatgaccggctctccggatccgaactacctggcagatctcatagcgactgtaaatgcaatcacccagaaaggtgcctacgccgtcgtcgatcctcataactacggcagatactacaattctataatctcgagcccttccgatttccagaccttctggaaaacggtcgcctcacagtttgcttcgaatccactggtcatcttcgacactaataacgaataccacgatatggaccagaccttagtcctcaatctcaaccaggccgctatcgacggcatccgttccgccggagccacttcccagtacatctttgtcgagggcaattcgtggaccggggcatggacctggacgaacgtgaacgataacatgaaaagcctgaccgacccatctgacaagatcatatacgagatgcaccagtacctggactctgacggatccgggacatcagcgacctgcgtatcttcgaccatcggtcaagagcgaatcaccagcgcaacgcagtggctcagggccaacgggaagaagggcatcatcggcgagtttgcgggcggagccaacgacgtctgcgagacggccatcacgggcatgctggactacatggcccagaacacagacgtctggactggcgccatctggtgggcggccgggccgtggtggggagactacatattctccatggagccggacaatggcatcgcgtatcagcagatacttcctattttgactccgtatctttga

SEQ ID NO: 20 - protein, 335 aa, Thermoascus aurantiacus
MKLGSLVLALSAARLTLSAPLADRKQETKRAKVFQWFGSNESGAEFGSQNLPGVEGKDYIWPDPNTIDTLISKGMNIFRVPFMMERLVPNSMTGSPDPNYLADLIATVNAITQKGAYAVVDPHNYGRYYNSIISSPSDFQTFWKTVASQFASNPLVIFDTNNEYHDMDQTLVLNLNQAAIDGIRSAGATSQYIFVEGNSWTGAWTWTNVNDNMKSLTDPSDKIIYEMHQYLDSDGSGTSATCVSSTIGQERITSATQWLRANGKKGIIGEFAGGANDVCETAITGMLDYMAQNTDVWTGAIWWAAGPWWGDYIFSMEPDNGIAYQQILPILTPYL

SEQ ID NO: 21 - DNA, 1520 bp, Talaromyces leycettanus
atggtccatctttcttccctggccctggctttggccgccggctcgcagctgtatgtgatccatgccatgactcgagaagtgctcccaaaactgactccaagtctcaatcttagtgcccaagctgcaggtcttaacactgctgccaaagcgattggaaagctctatttcggtaccgcaaccgacaacccggagctgtccgacagcacatacatgcaggagacggataacaccgatgatttcggccaactcaccccagctaactccatgaaggttcgctgacatcttagttcccccccccttttgggaatctgcgcggagatatgctgagccttcaaaactagtgggatgccaccgagccctctcagaacaccttcaccttcaccaacggtgatcagatcgcaaaccttgctaagagcaacggtcagatgctgagatgccacaacctggtgtggtacaaccagttgcccagctggggtaagcaaccggttctgttaatatcatcagcgtgaccgcatcgatcgtattgcgcggagattggaaagatttgcaagctaatgtcactacagtcaccagcggatcttggaccaatgccacgcttcttgcggccatgaagaaccacatcaccaacgttgtgacccactacaagggacagtgctacgcttgggatgttgtcaacgaaggtacgtttcgattcggcttccctcggaccgtatctgcaggcaaaaaggtcaatcaattgacaatcgtgatccccagctctcaacgatgatggcacctaccgatccaatgtcttctatcagtacatcggcgaggcatacattcccattgcctttgcgaccgctgccgccgccgatccaaacgcgaagctctactacaacgactacaacattgagtaccccggcgccaaggccaccgccgcccagaacatcgtcaagatggtcaaggcttacggcgcgaaaatcgacggtgtcggtctgcaatctcacttcatcgttggcagcacccctagccagagctcccagcagagcaacatggctgctttcaccgcgctcggcgtcgaggtcgccatcaccgaactggatatccgcatgacgttgccttccaccagtgctctcttggcccagcaatccaccgattaccagagcactgtgtcggcttgcgtgaacactccgaagtgcattggtatcaccctctgggactggaccgacaagtactcctgggttcccaacaccttctccggccaaggtgacgcctgcccctgggattctaactaccagaagaagcctgcctactacggtatcttgactgcgctcggaggcagcgcttccacctccaccaccaccactctggtgacctccaccaggacttcgactacgaccagcacttcggccacctccacgtctactggcgttgctcagcactggggccagtgcggtggtatcggctggacagggccgactacctgcgctagcccctacacctgccaggaactgaatccctactactaccagtgcctgtaa

SEQ ID NO: 22 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 23 - DNA, 2391 bp, Talaromyces emersonii; identical to SEQ ID NO: 17

SEQ ID NO: 24 - protein, 796 aa, Talaromyces emersonii; identical to SEQ ID NO: 18